(19) United States
(12) Patent Application Publication
(10) Pub. No.: US 2023/0011398 A1
(43) Pub. Date: Jan. 12, 2023

(54) COMBINATION THERAPY APPROACH TO ELIMINATE HIV INFECTIONS

(71) Applicant: THE METHODIST HOSPITAL SYSTEM, Houston, TX (US)

(72) Inventor: Jin WANG, Bellaire, TX (US)

(21) Appl. No.: 17/781,962

(22) PCT Filed: Dec. 4, 2020
(86) PCT No.: PCT/US20/63297

§ 371 Date: Jun. 2, 2022

Related U.S. Application Data
(60) Provisional application No. 62/943,446, filed on Dec. 4, 2019.

(51) Int. Cl. A61K 31/4196; A61P 31/18

(52) U.S. Cl. CPC A61K 31/4196; A61P 31/18

(57) ABSTRACT

By a Selective Elimination of Host Cells Capable of Producing HIV (SECH) approach, which includes a combination of latency reversal, blocking of new infections, inhibition of autophagy and induction of apoptosis, host cells harboring productive HIV infections can be cleared from a subject. Disclosed herein are methods for treating or inhibiting HIV in a subject, comprising a) reactivating latent HIV integrated into the genome of a cell infected with HIV in the subject, b) optionally administering to the subject an effective amount of a therapeutic agent to inhibit HIV infection, and c) administering to the subject an effective amount of a therapeutic agent to eliminate or reduce the number of cells containing replication-competent HIV. Kits for treating or inhibiting HIV in a subject are also disclosed.

### BACKGROUND

The acquired immunodeficiency syndrome (AIDS) is caused by HIV which infects and depletes CD4⁺ T cells in the patients. The RNA genome of HIV is reversely transcribed into DNA and integrated into the host genome, resulting in persistent infections that are difficult to eradicate. Combination antiretroviral therapy (cART) targeting different stages of the HIV replication cycle can efficiently inhibit active HIV infections and prevent the onset of AIDS. However, a stable HIV reservoir persists after treatments by cART. Continuous antiretroviral treatments may be necessary to prevent the burst of new viral production from the latent HIV reservoir. Broad-spectrum antibodies against HIV are effective in inhibiting viremia by neutralizing the virus. However, antibodies do not target the integrated HIV. Two leukemia patients infected by HIV have been reported to be cleared of the virus through regimens of total body irradiation or chemotherapy plus antibody-mediated depletion of lymphocytes, followed by transplantation of hematopoietic stem cells from homozygous CCR5Δ432 donors. However, a cure for HIV infections that is practical for the general population remains to be developed.

After antiretroviral therapy, persistent latent HIV is primarily found in CD4⁺ memory T cells that are quiescent and long-lived. Eradicating latently infected HIV, particularly in long-lived CD4⁺ memory T cells, is therefore critical for curing HIV infection. Antigen-specific lymphocytes are activated after encountering a pathogen and undergo significant cell proliferation. Most of these expanded lymphocytes are removed by programmed cell death, while some of these cells develop into long-lived and quiescent immune memory cells. As a major reservoir harboring latent HIV, quiescent memory T cells do not actively produce HIV and lack the display of viral antigens. They can thus evade the surveillance by the immune system and represent a major obstacle to eradicating HIV infections. Two important features of memory T cells, quiescence and longevity, are hijacked by HIV to maintain its latent and persistent infection. Re-activating the cells that are latently infected by latency reversal agents (LRAs) to display viral antigens may trigger immune responses against infected cells. However, LRAs alone have not been shown to reduce or clear HIV infections, showing the requirement for additional approaches. A new strategy is needed to target both quiescence and longevity of the host cells in order to eradicate the HIV reservoir.

HIV infections can trigger different cell death pathways in T cells. It has been shown that productive HIV-1 infections trigger caspase-dependent apoptosis in host cells, while abortive HIV-1 infections induce pyroptosis. Most integrated HIV type 1 (HIV-1) are defective, while only a small portion (<3%) of integrated HIV-1 are capable of producing infectious virions. A majority of integrated HIV-1 are non-productive that do not need to be cleared because they pose no risk for generating new infectious virions. To achieve a cure for HIV, it is necessary to deplete the reservoir harboring productive HIV infections, especially the infected memory T cells. However, the task of finding “a needle in a haystack” to seek out and destroy the HIV reservoir has been challenging.

What are needed are compositions and methods for targeting both quiescence and longevity of the host cells infected with HIV in order to eradicate the HIV reservoir. The compositions and methods disclosed herein address these and other needs.

### SUMMARY

By a Selective Elimination of Host Cells Capable of Producing HIV (SECH) approach, which includes a combination of latency reversal, blocking of new infections, inhibition of autophagy and induction of apoptosis, host cells harboring productive HIV infections can be cleared from a subject. Disclosed herein are methods for treating or inhibiting human immunodeficiency virus (HIV) in a subject, comprising a) reactivating latent HIV integrated into the genome of a cell infected with HIV in the subject, b) optionally administering to the subject an effective amount of a therapeutic agent to inhibit HIV infection, and c) administering to the subject an effective amount of a therapeutic agent to eliminate or reduce the number of cells containing replication-competent HIV. Treatment or inhibition of HIV can be determined by RT-PCR, virus outgrowth assay, intracellular staining for HIV p24, or the absence of virus rebound after withdrawal of the treatments, or the adoptive transfer of treated cells into humanized mice for virus outgrowth in vivo.

Step a), reactivating latent HIV, can comprise contacting the cell infected with HIV with a protein kinase activator; a glycogen synthase kinase 3 inhibitor; a bromodomain inhibitor; a histone deacetylase (HDAC) inhibitor; a histone acetyltransferase (HAT) inhibitor; phytohemagglutinin; a noncanonical NF-kB activator; an epigenetic modifier (e.g., JQ1D or CPI-203); a toll-like receptor (TLR) agonist; a cytokine; inhibitor of apoptosis (IAP) antagonist (IAP inhibitor e.g., AZD5582); or a combination thereof. In some embodiments, reactivating latent HIV comprises contacting the cell infected with HIV with a protein kinase activator, preferably a protein kinase C activator such as ingenol-3,20-dibenzoate; prostratin; bryostatin; a salt, ester, or prodrug thereof; or a combination thereof. In some examples, reactivating latent HIV comprises contacting the cell infected with HIV with ingenol-3,20-dibenzoate in an amount from 0.2 mg/kg to 10 mg/kg body weight, or from 0.2 mg/kg to 1.5 mg/kg body weight. In some embodiments, reactivating latent HIV comprises contacting the cell infected with HIV with a protein kinase activator and a bromodomain inhibitor. The protein kinase C activator can include ingenol-3,20-dibenzoate and the bromodomain inhibitor can include JQ1. The bromodomain inhibitor, preferably JQ1, can be used in an amount from 1 mg/kg to 100 mg/kg body weight or from 1 mg/kg to 50 mg/kg body weight.

In some embodiments of the methods disclosed herein, the method includes step b) administering a therapeutic agent to inhibit HIV infection. The therapeutic agent used in step b) to inhibit HIV infection can be selected from a therapeutic agent that inhibits HIV function. Such HIV function can include, but are not limited to, viral replication, viral protease activity, viral reverse transcriptase activity, viral entry into a cell, viral integrase activity, viral Rev activity, viral Tat activity, viral Nef activity, viral Vpr activity, viral Vpu activity, or viral Vif activity, or an anti-HIV broad neutralization antibody, an anti-HIV CAR T cell, or a combination thereof. In some embodiments, the therapeutic agent used to inhibit HIV infection can inhibit viral entry into cells (e.g., CD4⁺ T cells), inhibit viral integration into cells, or a combination thereof. Suitable therapeutic agents used to inhibit HIV infection can include BMS-626529 or its prodrug, BMS-663068 (BMS-663068 is the soluble preform of BMS-626529; BMS-663068 is more likely to be used orally); raltegravir; a salt, ester, or prodrug thereof; or a combination thereof. In some examples, the methods disclosed herein can include step b) administering to the subject BMS-626529 (or BMS-663068) in an amount from 2 mg/kg to 100 mg/kg body weight or from 5 mg/kg to 20 mg/kg body weight, to inhibit HIV infection. In further examples, the methods disclosed herein can include step b) administering to the subject raltegravir in an amount from 2 mg/kg to 100 mg/kg body weight or from 5 mg/kg to 20 mg/kg body weight, to inhibit HIV infection. In even further examples, step b) comprises administering both BMS-626529 (or BMS-663068) and raltegravir to the subject to inhibit HIV infection. The therapeutic agent used in step c) to eliminate or reduce the number of cells containing replication-competent HIV can be selected from a therapeutic agent that inhibits anti-apoptotic molecules (e.g., inhibits BCL-2); inhibits autophagy; or a combination thereof. Cells containing replication-competent HIV can include CD4⁺ T cells, macrophages, dendritic cells, or combinations thereof. In some examples, the therapeutic agent to eliminate or reduce the number of cells containing replication-competent HIV in step c) inhibits autophagy. As exemplified herein, inhibition of autophagy can simultaneously promote apoptosis. In some examples, the therapeutic agent to eliminate or reduce the number of cells containing replication-competent HIV in step c) inhibits anti-apoptotic molecules. For example, the therapeutic agent to eliminate or reduce the number of cells containing replication-competent HIV can comprise a BCL-2 inhibitor such as navitoclax (ABT-263) ABT-119, or venetoclax (RG7601 or GDC-0199); a MCL-1 inhibitor such as AZD5991, 563845, or AMG 176; SAR-405; chloroquine; or a combination thereof. In some examples, the methods disclosed herein can include step c) administering to the subject navitoclax in an amount from 2 mg/kg to 100 mg/kg body weight or from 5 mg/kg to 20 mg/kg body weight, to eliminate or reduce the number of cells containing replication-competent HIV. In further examples, the methods disclosed herein can include step c) administering to the subject SAR-405 in an amount from 2 mg/kg to 100 mg/kg body weight or from 5 mg/kg to 20 mg/kg body weight, to eliminate or reduce the number of cells containing replication-competent HIV. In even further some examples, step c) can comprise administering both navitoclax (ABT-263) and SAR-405 to the subject, to eliminate or reduce the number of cells containing replication-competent HIV.

In some aspects of the methods for treating or inhibiting human immunodeficiency virus (HIV) in a subject, the method can comprise administering IDB, ABT-263, and SAR405 or chloroquine. The therapeutic agents can be administered simultaneously, in sequence, or a combination thereof. For example, IDB, ABT-263, and SAR405 or chloroquine can be administered to a patient in need simultaneously. In other examples, at least two therapeutic agents (e.g., IDB and ABT-263) can be administered together to a patient in need followed by administering SAR405 or chloroquine. In further examples, IDB, ABT-263, and SAR405 or chloroquine can be administered at separate times to a patient in need.

The methods described herein for treating or inhibiting HIV can be repeated at least once every 2-7 days (such as every other day to once per week or longer intervals, every 2-3 days, once every 2 days (every other day)). In these embodiments, the methods can be repeated continuously for at least 6 weeks, at least 7 weeks, at least 8 weeks, from 6 to 40 weeks, or from 6 to 10 weeks. The therapeutic agents can be administered to the subject orally and/or intravenously.

The methods disclosed herein for treating or inhibiting human immunodeficiency virus (HIV) in a subject can kill 95% or greater (for e.g., 97% or greater, 99% or greater, or up to 100%) of infected cells within 2 days. Advantageously, uninfected cells are substantially resistant to apoptosis using the methods disclosed herein. In particular, 90% or greater (for e.g., 92% or greater, 94% or greater, 95% or greater, 97% or greater, 97% or greater, 99% or greater, or up to 100%) of uninfected cells are resistant to apoptosis using the methods disclosed herein.

Kits for treating or inhibiting HIV in a subject are also disclosed. The kits can include an effective amount of a therapeutic agent for reactivating latent HIV integrated into the genome of a cell infected with HIV in the subject, an effective amount of a therapeutic agent to inhibit HIV infection in the subject, and an effective amount of a therapeutic agent to eliminate or reduce the number of cells containing replication-competent HIV in the subject. For example, the kits disclosed herein can include a first component comprising ingenol-3,20-dibenzoate, JQ1, or a combination thereof; a second component comprising BMS-626529 (or its prodrug BMS-663068), raltegravir, or a combination thereof; and a third component comprising navitoclax (ABT-263), venetoclax (ABT-199) or other chemicals targeting BCL-2 family members, SAR-405, chloroquine, or a combination thereof.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects described below.

FIGS. 1A-1I show regulation of host cell survival, but not HIV-1 reverse transcription and integration into the host genome by inhibition of autophagy. (FIG. 1A) CMT were transfected with Atg7 siRNA, infected with HIV-1 (NL4-3, 0.1 MOI) and cultured for 4 d to establish latency. After latency reversal by PHA, the percentage of p24+ cells (n=3 biologically independent samples) and Atg7 protein (representative of two biologically independent experiments) were determined. *p=0.011. (FIG. 1B) CMT latently infected with HIV-1 were stimulated with IDB with or without SAR405 (2 μM) or CQ (10 μM). The percentage of p24+ cells (n=3 biologically independent experiments). LC3 punctate per cells (n=30 cells for each group from two biologically independent experiments) are shown. p24+cells: p=0.010 (SAR405) and 0.0098 (CQ); LC3 punctates/cell, 0.0001 (no virus) and 0.0001 (HIV-1). (FIGS. 1C, 1D) CMT transfected with Atg7 siRNA were infected with HIV-1. Alternatively, CMT infected with HIV-1 were cultured with or without SAR405. Cells were collected at different time after infection to perform RT-PCR for R/U5 (1C) and LTR-gag (1D) (n=3 biologically independent samples). (FIG. 1E) Genomic DNA were extracted from CMT as in (1A), CMT cultured with SAR405 as in (1B) or infected CMT for Alu-gag PCR. Data were normalized against β-Globin. ND: not detectable. Data are presented as mean ±SD (n=3 biologically independent samples). (FIG. 1F) T cells latently infected and cultured as in (1A) and (1B) were reactivated with PHA for 24 h. HIV-1 mRNA was determined by RT-PCR. Data are presented as mean ±SD (n=3 biologically independent samples). The dash line indicates detection limit.(FIG. 1G) HIV-1 mRNA in PBMCs from ART-treated HIV-1-infected patients were stimulated with IDB in the presence or absence of SAR405 for 24 h (n=5 patients). (FIGS. 1H, 1I) CMT were latently infected with HIV-1 (NL4-3, 1 MOI) as in (1A). The cells were reactivated with 100 nM IDB for 24 h. Percentages of cells positive for annexin V (1H) or DEVD (1I) staining were analyzed by flow cytometry (n=3 biologically independent samples). SAR405 vs. control, p=0.0012 (h), 0.0024 (i); CQ vs. control, p=0.0164 (h), 0.0088 (1I).

FIGS. 2A-2E shows induction of caspase activation and cell death in HIV-1-infected T cells. (FIG. 2A) CD4⁺ T cells from PBMCs with or without infection by HIV-1 (NL4-3, 1 MOI) were cultured for 4 days to establish latency, followed by stimulation with IDB for 24 h. Cell lysates were used for Western blot (representative of two biologically independent experiments). Arrows indicate cleaved caspases. (FIG. 2B) CMT with or without infection by HIV-1 (NL4-3, 1 MOI) were cultured for 4 days to establish latency. The cells were stimulated with 0.1 μM IDB. ABT-263 (0.2 μM) and SAR405 (2 μM) and chloroquine (CQ, 10 μM) were added as indicated. The cells were cultured for 48 h, followed by incubation with DEVD-FITC, staining with APC-Annexin V and intracellular staining with PE-anti-HIV p24. (FIG. 2C) Total cell death for cells treated in (2B) was calculated (n=3 biologically independent samples). Data are presented as mean ±SD. p values for control vs. five treatment groups in sequence: 0.0001, 0.0011, 0.0002, 0.0049 and 0.0001 (one-way ANOVA with unpaired two-tailed t-test). (FIG. 2D) The combinations of ABT-263 and SAR405 or CQ in the killing of IDB-stimulated HIV-1-infected T cells in (b) was calculated. The remaining viable HIV-1 p24⁺ cells (negative for staining by APC-annexin V and DEVD-FITC) in (b) were also calculated. Data are presented as mean ±SD (n=3 biologically independent samples). p values for control vs. 4 treatment groups in sequence (for Killing of p24⁺cells and for p24⁺cells remaining): 0.0001, 0.0006, 0.0001, 0.0011 and 0.0001 (One-way ANOVA with unpaired two-tailed t-test). (FIG. 2E) T cells latently infected with HIV-1 as in FIG. 1A were mixed with CellTrace Violet-labeled uninfected T cells, and cultured with IDB, SAR405 and ABT263 as in (FIG. 2B), in the presence of 0.2 BMS-626529 for 48 h. The cells were stained with DEVD-FITC and APC-Annexin V, followed by flow cytometry analysis. Data are presented as mean ±SD (n=3 biologically independent samples). p values for control vs. 4 treatment groups in sequence: 0.0059, 0.0005, 0.0004 and 0.0001 (One-way ANOVA with unpaired two-tailed t-test).

FIGS. 3A-3G show treatment of HIV-1 infections in Hu-HSC mice by SECH. (FIG. 3A) The SECH regimen includes: 1) Latency reversion; 2) Induction of cell death; 3) Inhibition of autophagy; and 4) Blocking of new infections with inhibitors for HIV-1 attachment and integration. (FIG. 3B) An example of flow cytometry analyses of human cells in the peripheral blood of NSG-SGM3 mice 3 months after reconstitution with human CD34⁺ stem cells. Cell negative for mouse CD45 (mCD45) and positive for human CD45 (hCD45) were gated to analyze CD19⁺ human B cells, CD3⁺CD4⁺ and CD3⁺CD8⁺ human T cells. (FIG. 3C) Three months after reconstitution with human CD34⁺ stem cells, one set of HIV-1-infected Hu-HSC mice (FIG. 17) were infected with HIV-1 (AD8 strain, 1000 pfu/mouse) intraperitoneally. Ten days after HIV-1 infections, the mice were used for treatments by ART or SECH. (FIGS. 3D, 3E) RNA from the whole blood (including plasma and cells) was extracted to measure HIV-1 mRNA in mice treated by SECH (FIG. 3E) or ART (FIG. 3F) for 40 cycles. The dash line indicates detection limit. (FIG. 3F) HIV-1 mRNA in the spleen and bone marrow of mice treated by SECH or ART was determined by RT-PCR. Data are presented as mean ±SD (n=3 technical replicates). ND, not detectable. (FIG. 3G) Infectious HIV-1 in the spleen and bone marrow of mice treated by SECH or ART was measured by TZA assays. Data are presented as mean ±SD (n=3 technical replicates).

FIGS. 4A-4C show clearance of HIV-1 infections in Hu-HSC mice by SECH. (FIG. 4A) HIV-1 viral RNA levels in the blood of SECH- or ART-treated Hu-HSC mice before and after 2 months of withdrawal of the treatments (ART, n=5; SECH, n=15). The dash line indicates detection limit. (FIG. 4B) Measuring infectious HIV-1 from spleen and bone marrow (BM) cells of HIV-1-infected Hu-HSC mice by TZA. Data are presented as mean ±SD (ART, n=5 mice; SECH, n=15 mice). Data are presented as mean ±SD. Each biological sample was measured in three technical replicates. ND, not detectable. (FIG. 4C) Virus outgrowth assay after adoptive transfer of spleen and BM cells from mice into uninfected Hu-HSC recipient mice. (ART, n=3 mice; SECH, n=15 mice).

FIGS. 5A-5D show improvement of SECH treatment by inclusion of JQ1. (FIG. 5A) Viral RNA levels in one set of HIV-1-infected Hu-HSC mice (FIG. 19) orally treated by SECH with (black symbols) or without JQ1 (red symbols) for a total of 35 cycles (ART, n=5 mice; SECH, n=10 mice; SECH +JQ1, n=13 mice). The dash line indicates detection limit. (FIG. 5B) Viral titer in the blood before and after withdrawal of the treatments. (SECH, n=10 mice; SECH +JQ1, n=13 mice). No JQ1 vs. JQ1, p=0.0407 (Mann-Whitney test). (FIG. 5C) Measuring infectious HIV-1 from spleen and bone marrow cells of HIV-1-infected Hu-HSC mice by TZA. Biological samples from each mouse were measured in three technical replicates. Data are presented as mean ±SD (ART, n=5 mice; SECH, n=10 mice; SECH +JQ1, n=13 mice). ND, not detectable. (FIG. 5D) hmVOA for spleen and bone marrow cells from mice in (c) (SECH, n=7 mice; SECH +JQ1, n=13 mice).

FIGS. 6A-6D shows clearance of HIV-1-infected cells from ART-naïve HIV-1 patients by SECH treatments in vitro. (FIG. 6A) CD4 counts and HIV-1 load in ART-naïve HIV-1-infected patients (n=10) without previous antiretroviral treatment. A, Asian; B, Black; C, Caucasian. (FIG. 6B) PBMCs obtained from ART-naïve HIV-1 patients (n=10) were culture in vitro with agents for SECH or ART only for 2 days as on cycle, with a total of 5 cycles as described in the Method section. Samples were also re-stimulated with IDB for detection of HIV-1 mRNA by RT-PCR. The dash line indicates detection limit. (FIG. 6C) PBMCs treated by in (FIG. 6B) were depleted of CD8⁺ T cells and 3×10⁶ cells were adoptively transferred into uninfected Hu-HSC mice for detection of HIV-1 by hmVOA. (FIG. 6D) The cells treated in (FIG. 6B) were used for TZA analyses. Ten biological samples were each measured in three technical replicates and presented as mean ±SD.

FIGS. 7A-7D show clearance of HIV-1-infected cells from ART-experienced HIV-1 patients by SECH treatments in vitro. (FIG. 7A) CD4 counts and HIV-1 load in ART-experienced HIV-1-infected patients (n=10). HIV viremia was successfully suppressed in all patients at the time of blood collection. Patients 18 and 19 had undergone ART treatments for 139 and 95 days, respectively. (FIGS. 7B-7D) PBMCs obtained from ART-experienced HIV-1-infected patients (n=10) were treated in vitro by SECH or ART only for 5 cycles, and then used for detection of HIV-1 mRNA by RT-PCR (FIG. 7B) hmVOA (FIG. 7C) and TZA analyses (FIG. 7D) as in FIG. 6. Ten biological samples were each measured in three technical replicates and presented as mean ±SD (FIG. 7D).

FIGS. 8A-8C show establishment of HIV-1 latent infection in sorted T cell. (FIG. 8A) CD4⁺ T cells were purified from human PBMCs using anti-CD4-MACS beads (Miltenyi Biotec). CD3⁺CD4⁺CD45RA⁺CD45RO⁻CCR7⁺ naive T cells, CD3⁺CD4⁺CD45RA⁻CD45RO⁺CCR7⁺ central memory T cells (CMT) and CD3⁺CD4⁺CD45RA⁻CD45RO⁺CCR7⁻effector memory T cells (EMT) were sorted by flow cytometry. (FIG. 8B) CMT were infected with HIV-1 and cultured for 4 days with CCL19 to establish latent infections. Latently infected cells were stimulated with PHA for virus reactivation, followed by intracellular staining for HIV-1 p24. (FIG. 8C) Latently infected cells were stimulated with PHA or IDB for virus reactivation, followed by pol RT-PCR analyses. Data are presented as mean ±SD (n=3 biologically independent samples). The dash line indicates detection limit.

FIGS. 9A-9B show inhibition of autophagy in HIV-1-infected T cells. (FIG. 9A) CMT with or without HIV-1 infection as in FIG. 1 were cultured with or without SAR405 for 12 h, following by immunocytochemistry staining for LC3. The images are representative of two independent experiments. Scale bar, 10 mm. (FIG. 9B) CMT were infected with HIV-1 AD8 (1 MOI). The cells were cultured with CCL19 in the presence or absence of SAR405 for 4 days. The cells were then cultured with or without IDB for 24 h, followed by intracellular staining for HIV-1 p24 and flow cytometry. Percentages of HIV-1 p24⁺ cells are shown.

FIGS. 10A-10D show induction of cell death in T cells with or without HIV-1 infections. (FIG. 10A) Naive T cells, activated T cells, or EMT with or without infection by HIV-1 (NL4-3, 1 MOI) were cultured for 4 days to establish latency as in FIG. 1. The cells were stimulated with 0.1 μM IDB. ABT-263 (0.2 μM), SAR405 (2 μM) and chloroquine (CQ, 10 μM) were added in different samples as indicated. The cells were cultured for 48 h. The cells were then incubated with DEVD-FITC, followed by staining with APC-Annexin V and intracellular staining with PE-anti-HIV p24. The cells were analyzed by flow cytometry. (FIG. 10B) Percentage of cell death among all T cells. (FIG. 10C) Percentage of killing of HIV-1 p24⁺ T cells by indicated combination of agents after IDB-induced virus reactivation. (FIG. 10D) Percentage of remaining HIV-1 p24⁺ viable T cells negative for DEVD-FITC and Annexin V staining after treatments by indicated combinations of ABT-263 and SAR405 or CQ compared to untreated controls. In FIGS. 10B, 10C and 10D, data are representative of three biologically independent samples and presented as mean ±SD.

FIGS. 11A-11C show analyses of Hu-HSC mice by flow cytometry. (FIG. 11A) Flow cytometry analyses of immune cells in the peripheral blood of Hu-HSC mice. NK, nature killer cells; DC, dendritic cells; M1, M1 macrophages; M2, M2 macrophages. (FIG. 11B) Spleen cells were isolated from Hu-HSC mice (n=4) 10 days post HIV-1 infection and cultured with or without PHA for 24 h. Culture media were collected for HIV-1 mRNA determination by RT-PCR. Data are presented as mean ±SD. PHA vs. control, **p=0.0088 (unpaired two-tailed t test). (FIG. 11C) Spleen cells treated in (b) were stained for CD4, followed by intracellular staining of HIV-1 p24 and flow cytometry analysis. Increases in p24⁺ cells after PHA stimulation suggest that HIV-1 infections in some cells have established latency. Data are presented as mean ±SD. PHA vs. control, **p=0.0003 (unpaired two-tailed t test).

FIGS. 12A-12G show SECH treatment in HIV-1-infected Hu-HSC mice. (FIG. 12A) Detection of HIV-1 mRNA in tissue and organs from mice after treatments by SECH or ART. (FIG. 12B) Determination of HIV-1 genomic DNA in spleen from mice after treatments by SECH (n=5) or ART (n=5). Data are presented as mean ±SD. SECH vs. ART: **p=0.0001 (unpaired two-tailed t test). (FIG. 12C) HIV-1 mRNA in the total peripheral blood was determined by pol RT-PCR prior to SECH treatment. The dash line indicates detection limit. (FIGS. 12D, 12E) HIV-1 mRNA in the total peripheral blood was determined by pol RT-PCR after SECH (FIG. 12D) or ART (FIG. 12E) treatment. (FIG. 12F) HIV-1 mRNA in the peripheral blood of HIV-1-infected Hu-HSC mice after withdrawal of treatments for 4 weeks. (FIG. 12G) Infectious HIV-1 in the spleen of mice in (f) was determined by TZA assays. ND, not detectable. In FIGS. 12A and 12G, biological samples were measured in three technical replicates and data are presented as mean ±SD. Source data are provided as a Source Data file.

FIGS. 13A-13C show analyses of Hu-HSC mice treat by SECH or ART. (FIG. 13A) Detection of HIV-1 p24 in T cells from Hu-HSC mice after SECH treatments. Spleen cells of SECH-treated mice were stimulated with PHA plus LPS and CpG. mCD45⁻hCD19⁻hCD45⁺CD3⁺CD4⁺human CD4 T cells were gated to determine p24 staining. Human PBMC CD4⁺ T cells with or without HIV-1 infection were used as controls. (FIG. 13B) Mouse body weight during SECH treatment in FIGS. 3 and 4. (FIG. 13C) H&E staining of tissue sections from HIV-infected Hu-HSC mice treated by ART or SECH (representative of 4 biologically independent samples for each treatment) . Scale bar: 200 μm.

FIGS. 14A-14B show analyses of Hu-HSC mice treated by SECH or ART. (FIG. 14A) HIV-1-infected Hu-HSC mice were treated by SECH or ART for 40 cycles. After withdrawal of treatments, the types of immune cells from the spleen were determined by flow cytometry (left panel). The number of immune cells in the spleen were shown in right panel (ART, n=5; SECH, n=10). Data are presented as mean ±SD. (FIG. 14B) HIV-1-infected Hu-HSC mice were treated by SECH or ART for 40 cycles. After withdrawal of treatments, immunological memory cells from the spleen were determined flow cytometry (left panel). CCR7⁺CCD45RO⁻ naïve T cells, CCR7⁺CCD45RO⁺central memory T cells, CCR7⁻CCD45RO⁺effector memory T cells and CCR7⁻CCD45RO⁻activated effector T cells were examined. CD19⁺CD27⁺memory B cells were also quantified (right panel). Data are presented as mean ±SD. SECH (n=5) vs. ART (n=3): p=0.0111 (Naïve T), 0.0006 (Central memory T cells), 0.0111 (Effector and effector memory T) and 0.4627 (Memory B cells) as determined by unpaired two-tailed t test.

FIG. 15A-15D show SECH treatment in HIV-1-infected Hu-HSC mice. (FIG. 15A) Latently infected CD4 T cells were cultured with IDB, JQ1 or both for 24 h. HIV-1 mRNA was measured by RT-PCR. Data are presented as mean ±SD (n=3 biologically independent samples). (FIG. 15B) Mouse body weight during SECH treatments in FIG. 5. (FIG. 15C) H&E staining of tissue sections from HIV-infected Hu-HSC mice treated by ART or SECH. Data are representative of 4 biologically independent samples for each treatment. Scale bar: 200 μm. (FIG. 15D) Flow cytometry analyses of immune cells in the spleen of Hu-HSC mice treated by ART (n=4), SECH (n=8) or SECH+JQ1 (n=8). Data are presented as mean ±SD.

FIGS. 16A-16F show SECH treatment in patient samples. (FIG. 16A) Determination of HIV-1 mRNA in culture media from PBMCs of ART-treated patients (n=4) by pol RT-PCR. (FIG. 16B) PBMCs of ART-treated patients were treated by ART, SECH, SECH without antivirus drugs, respectively, for 5 cycles, cell associated HIV-1 mRNA was determined by pol RT-PCR. (FIG. 16C) The percentage of cell survival and loss of live cells after one cycle of treatments by SECH or ART(n=3 biologically independent experiments). Data are presented as mean ±SD. (FIG. 16D) The percentage of immune cells in PBMCs from ART-treated patients (n=10) after treatment with ART or SECH. Data are presented as mean ±SD. (FIG. 1E) CD19⁺IgD⁻CD27⁺memory B cells in PBMCs from HIV-1⁺patients after 1 cycle of treatment by ART or SECH. Data are presented as mean ±SD. (FIG. 16F) PBMCs purified from HIV-1⁺patients (n=4) were treated by SECH or ART for 5 cycles. T cells were determined by flow cytometry. Data are presented as mean ±SD. SECH vs. ART: p=0.0060 (Naive T cells), 0.5502 (Central memory T cells) and 0.0001 (Effector memory and effector T cells) as determined by unpaired two-tailed t test.

FIG. 17 is a table showing HIV-1-infected Hu-HSC mice used for SECH treatments.

FIG. 18 is a table showing testing of HIV-1 infections and treatment using Hu-HSC mice.

FIG. 19 is a table showing Hu-HSC mice used for SECH+JQ-1 treatments for HIV-1 infections.

### DETAILED DESCRIPTION

The materials, compositions, kits, and methods described herein may be understood more readily by reference to the following detailed description of specific aspects of the disclosed subject matter, the Figures, and the Examples included therein. Before the present materials, compositions, kits, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the transplant” includes mixtures of two or more such transplants, and the like.

“Optional” or “optionally” means that the subsequently described event or circumstance can or cannot occur, and that the description includes instances where the event or circumstance occurs and instances where it does not.

The term “subject” refers to any individual who is the target of administration or treatment. The subject can be a vertebrate, for example, a mammal. Thus, the subject can be a human or veterinary patient. In some embodiments, the subject is a human. The term “patient” refers to a subject under the treatment of a clinician, e.g., physician. The term “disease” refers to a state of health of a subject wherein the subject cannot maintain homeostasis, and wherein if the disease is not ameliorated then the subject's health continues to deteriorate. The term “disorder” refers to a state of health in which the subject is able to maintain homeostasis, but in which the subject's state of health is less favorable than it would be in the absence of the disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

A “pharmaceutically acceptable” component is one that is suitable for use with humans and/or animals without undue adverse side effects (such as toxicity, irritation, and allergic response) commensurate with a reasonable benefit/risk ratio.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Examples of prodrugs that can be used to improve bioavailability include esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, L-amino acid esters, D-amino acid esters, N-substituted L-amino acid esters, N,N-disubstituted L-amino acid esters, N-substituted D-amino acid esters, N,N-disubstituted D-amino acid esters, sulfenyl, optionally substituted sulfenyl, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted to thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, bis-(acyloxybenzyl)esters, optionally substituted bis-(acyloxybenzyl)esters, (acyloxybenzyl)esters, optionally substituted (acyloxybenzyl)esters, and BAB-esters. As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

“Pharmaceutically acceptable derivative” or “pharmaceutically acceptable salt” refers to a prodrug or salt that is pharmaceutically acceptable and has the desired pharmacological properties. Such derivatives or salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

As used herein, the term “substantially” refers to the complete or nearly complete extent or degree of an action, characteristic, property, state, structure, item, or result. For example, where uninfected cells are “substantially” resistant to apoptosis using the disclosed methods would mean that the unaffected cells are either completely or nearly completely intact after performing the methods disclosed herein.

Reference will now be made in detail to specific aspects of the disclosed materials, therapeutic agents, compositions, and methods, examples of which are illustrated in the accompanying Examples.

### Methods

Disclosed herein are methods for treating or inhibiting human immunodeficiency virus (HIV) in a subject, by a Selective Elimination of Host Cells Capable of Producing HIV (SECH) approach. The SECH approach combines latency reversal, blocking of new infections, inhibition of autophagy and induction of apoptosis, to clear host cells harboring productive HIV infections. In some embodiments, the methods for treating or inhibiting HIV comprise a) reactivating latent HIV integrated into the genome of a cell infected with HIV in the subject, b) optionally administering to the subject an effective amount of a therapeutic agent to inhibit HIV infection, and c) administering to the subject an effective amount of a therapeutic agent to eliminate or reduce the number of cells containing replication-competent HIV. Treatment or inhibition of HIV can be determined by RT-PCR, virus outgrowth assay, intracellular staining for HIV p24, and the absence of virus rebound after withdrawal of the treatments.

The step of reactivating latent HIV integrated into the genome of an HIV-infected cell (step a)) can reduce the reservoir of latent immunodeficiency virus in vitro and in vivo. In some embodiments, the step of reactivating latent HIV (step a)) can comprise contacting the cell in which HIV is latent with a compound that activates one or more of the different isoforms of protein kinase, such as a protein kinase C activator. In other embodiments, the step of reactivating latent HIV (step a)) can comprise contacting the cell in which HIV is latent with a compound that suppresses or inhibits the action of one or more of the different isoforms of the serine/threonine kinase glycogen synthase kinase 3, such as for example a glycogen synthase kinase 3 inhibitor or a glycogen synthase kinase 313 inhibitor; a bromodomain inhibitor; a histone deacetylase (HDAC) inhibitor; a histone acetyltransferase (HAT) inhibitor; phytohemagglutinin; a noncanonical NF-kB activator; an epigenetic modifier (e.g., JQ1D or CPI-203); a toll-like receptor (TLR) agonist; a cytokine; an inhibitor of apoptosis (IAP) antagonist (IAP inhibitor e.g., AZD5582); or a combination thereof.

In some embodiments, the step of reactivating latent HIV in a cell can reduce the reservoir of latent HIV in a subject by contacting the cell with an effective amount of a protein kinase activator. The protein kinase activator can be selected from ingenol-3,20-dibenzoate; prostratin; bryostatin; a pharmaceutically acceptable salt, ester, or prodrug thereof; or a combination thereof. In some embodiments, reactivating latent HIV comprises contacting the cell infected with HIV with a protein kinase activator and a bromodomain inhibitor. The protein kinase C activator can include ingenol-3,20-dibenzoate and the bromodomain inhibitor can include JQ1. In some embodiments, an effective amount of the protein kinase activator (e.g., a ingenol-3,20-dibenzoate or a pharmaceutically acceptable salt or derivative thereof) or protein kinase activator and bromodomain inhibitor can be in amount that reactivates latent HIV and reduces the reservoir of latent HIV in a subject by at least about 20%, at least about 30%, at least about 40%, at least about 50%, at least about 60%, at least about 70%), at least about 80%, or at least about 90%. A “reduction in the reservoir of latent HIV” (also referred to as “reservoir of latently infected cells”) is a reduction in the number of cells in the individual that harbor a latent HIV infection. Whether the reservoir of latently infected cells is reduced can be determined using any known method, including the method described in Blankson et al. (2000) J. Infect. Disease 182(6): 1636-1642.

In some examples, the methods disclosed herein can include contacting an HIV-infected cell in which HIV is latent with an effective amount of ingenol-3,20-dibenzoate. In some examples, the methods disclosed herein can include contacting an HIV-infected cell in which HIV is latent with an effective amount of ingenol-3,20-dibenzoate and JQl. An effective amount of ingenol-3,20-dibenzoate in a single dosage form, can be from 0.2 mg/kg to 10 mg/kg body weight or from 0.2 mg/kg to 1.5 mg/kg body weight, of the subject. An effective amount of JQ1 in a single dosage form, can be from 1 mg/kg to 100 mg/kg body weight or from 1 mg/kg to 50 mg/kg body weight, of the subject.

As described herein, the methods for treating or inhibiting HIV in a subject can optionally comprise step b) administering to the subject an effective amount of a therapeutic agent to inhibit HIV infection. In some embodiments, the method includes step b) administering a therapeutic agent to inhibit HIV infection. In particular, once the latent HIV cells are reactivated, the subject can be administered antiretroviral therapy to prevent or reduce the level of repopulation of the HIV reservoir. The antiretroviral therapy administered can be in an effective amount to inhibit an HIV function. The HIV function can be selected from viral replication, viral protease activity, viral reverse transcriptase activity, viral entry into a cell, viral integrase activity, viral Rev activity, viral Tat activity, viral Nef activity, viral Vpr activity, viral Vpu activity, and viral Vif activity. Other approaches to replace the pRTA portion in the SECH approach can be used as antiretroviral therapy, such as the use of anti-HIV broad neutralization antibodies or anti-HIV CAR T cells to replace pART agents in the SECH approach.

In some embodiments, the antiretroviral drug is selected from nucleoside-analog reverse transcriptase inhibitors (NRTIs). NRTIs refer to nucleosides and nucleotides and analogues thereof that inhibit the activity of HIV-1 reverse transcriptase. An example of nucleoside-analog reverse transcriptase inhibitors is, without limitation, adefovir dipivoxil. In some embodiments, the antiretroviral drug is selected from non-nucleoside reverse transcriptase inhibitors (NNRTIs). NNRTIs are allosteric inhibitors which bind reversibly at a nonsubstrate-binding site on the HIV reverse transcriptase, thereby altering the shape of the active site or blocking polymerase activity. Examples of NNRTIs include, without limitation, delavirdine (BHAP, U-90152; RESCRIPTOR™), efavirenz (DMP-266, SUSTIVA™), nevirapine (VIRAMUNE™), PNU-142721, capravirine (S-1153, AG-1549), emivirine (+)—calanolide A (NSC-675451) and B, etravirine (TMC-125), rilpivi ne (T C278, EDURANT™), DAPY (TMC120), BILR-355 BS, PHI-236, and PHI-443 (TMC-278). In some embodiments, the antiretroviral drug is selected from protease inhibitors (PI). Protease inhibitors are inhibitors of the HIV-1 protease. Examples of protease inhibitors include, without limitation, darunavir, amprenavir (141W94, AGENERASE™), tipranivir (PNU-140690, AP IVUS™), indinavir (MK-639; CRIXIVAN™), saquinavir (INVIRASE™, FORTOVASE™), fosamprenavir (LEXIVA™), lopinavir (ABT-378), ritonavir (ABT-538, NORVIR™), atazanavir (REYATAZ™), nelfinavir (AG-1343, VIRACEPT™), lasinavir (BMS-234475 /CGP-61755), BMS-2322623, GW-640385X (VX-385), AG-001859, and SM-309515. In some embodiments, the antiretroviral drug is selected from fusion inhibitors (FI). Fusion inhibitors are compounds, such as peptides, which act by binding to HIV envelope protein and blocking the structural changes necessary for the virus to fuse with the host cell. Examples of fusion inhibitors include, without limitation, maraviroc (SELZENTRY™, Celsentri), enfuvirtide (INN, FUZEON™), T-20 (DP-178, FUZEON™) and T-1249. In some embodiments, the antiretroviral drug is selected from integrase inhibitors. Integrase inhibitors are a class of antiretroviral drug designed to block the action of integrase, a viral enzyme that inserts the viral genome into the DNA of the host cell. Examples of integrase inhibitors include, without limitation, raltegravir, elvitegravir, and MK-2048. In some embodiments, the antiretroviral drug also include HIV vaccines such as, without limitation, ALVAC™ HIV (vCP1521), AIDSVAX™ B/E (gp120), and combinations thereof Anti-HIV compounds also include HIV antibodies (e.g., antibodies against gp120 or gp41), particularly broadly neutralizing antibodies. More than one anti-HIV agent may be used, particularly where the agents have different mechanisms of action (as outlined above). Specific examples of suitable antiretroviral drugs that can be used include one or more of the following antiretroviral compounds: lamivudine, ziduvudine, emtricitabine, abacavir, abacavir sulfate, zidovudine, tenofovir, didanosine, stavudine, delavirdine, efavirenz, nevirapine, etravirine, maraviroc, rilpivirine, raltegravir, atazanavir, efavirenz, indinavir, ritonavir, saquinavir, nelfinavir, amprenavir, lopinavir, fosamprenavir, tipranavir, darunavir, nelfinavir, brecanavir, boceprevir, TMC435, and declatasvir.

In some examples, an antiretroviral therapy can include any appropriate anti-retroviral agent or combination of anti-retroviral agents. In certain embodiments, the therapeutic agent to inhibit HIV infection can include an HIV integrase inhibitor such as raltegravir (also known as Isentress or MK-0518), dolutegravir, and elvitegravir. In certain embodiments, the therapeutic agent to inhibit HIV infection can include an HIV protease inhibitor such as lopinavir, atazanavir, Agenerase, Crixivan, Fortovase, Invirase, Kaletra, Norvir, Viracept, or a combination thereof. In certain embodiments, the therapeutic agent to inhibit HIV infection can include a reverse transcriptase inhibitor such as emtricitabine, rilpivirine, tenofovir, combivir, epivir, hivid, retrovir, videx, zerit, ziagen, or a combination thereof. In certain embodiments, the therapeutic agent to inhibit HIV infection can include a non-nucleoside reverse transcriptase inhibitor such as Rescriptor, Sustiva, Viramune, or a combination thereof. In certain embodiments, the therapeutic agent to inhibit HIV infection can include a therapeutic agent that inhibits viral entry into cells (e.g., CD4⁺ T cells) such as BMS-626529. BMS-663068 is the soluble preform of BMS-626529. BMS-663068 is more likely to be used orally. In certain embodiments, the therapeutic agent to inhibit HIV infection can include both an HIV integrase inhibitor and an agent that inhibits viral entry into cells (e.g., CD4⁺ T cells). For example, the therapeutic agent to inhibit HIV infection can be selected from BMS-626529 (or BMS-663068); raltegravir; a salt, ester, or prodrug thereof or a combination thereof. In some cases, combinations of antiretroviral agents can be formulated into a single dosage form (e.g., a single pill or capsule). Alternately, the therapeutic agents can be formulated as a multi dosage form, and the multi dosage formulation administered to a subject.

The methods disclosed herein for treating or inhibiting HIV in a subject can further comprise step c) administering to the subject an effective amount of a therapeutic agent to eliminate or reduce the number of cells containing replication-competent HIV. In some examples, the therapeutic agent to eliminate or reduce the number of cells containing replication-competent HIV in step c) inhibits autophagy. As exemplified herein, inhibition of autophagy can simultaneously promote apoptosis. In some examples, the therapeutic agent to eliminate or reduce the number of cells containing replication-competent HIV in step c) inhibits anti-apoptotic molecules. For example, the therapeutic agent to eliminate or reduce the number of cells containing replication-competent HIV can include a BCL-2 inhibitor or a MCL-1 inhibitor. Any appropriate BCL-2 inhibitor, MCL-1 inhibitor, or combination thereof (e.g., a combination of two, three, four, five, or more different BCL-2 inhibitors and/or MCL-1 inhibitors) can be used as described herein. Examples of BCL-2 inhibitors that can be used include, without limitation, ABT-199, ABT263, venetoclax (RG7601 or GDC-0199), and Sabutoclax. Examples of MLC-1 inhibitors that can be used include, without limitation, AZD5991, 563845, or AMG 176.

In some cases, one or more agents that facilitate cell death can be used in place of or in combination with the Bcl-2 inhibitor to treat HIV infections as described herein. For example, one or more agents that facilitate cell death by inhibiting autophagy (e.g., SAR-405 or chloroquine) can be used alone or in combination with one or more Bcl-2 inhibitors. In some cases, the therapeutic agent administered in step c), such as the Bcl-2 inhibitor and/or autophagy inhibitor, can be used to cause latent HIV infected cells to die following HIV reactivation in those latently HIV infected cells.

Accordingly, the present disclosure provides methods of treating an HIV in a subject comprises co-administering to the individual a therapeutic agent that reactivates latent HIV such as a protein kinase activator; administering an antiretroviral HIV agent therapeutic agent, and administering an HIV agent that facilitates cell death. In some embodiments, a protein kinase activator alone or in combination with a bromodomain inhibitor (e.g., ingenol-3,20-dibenzoate, JQ1, a pharmaceutically acceptable salt or derivative thereof, or a combination thereof) is administered in a combination therapy (i.e., co-administered) with: 1) one or more antiretroviral agents (e.g., viral entry inhibitor such as BMS-626529 or its prodrug BMS-663068) and HIV integrase inhibitor such as raltegravir); and 2) one or more Bcl-2 inhibitor such as ABT263 or MCL-1 inhibitor, and optionally an autophagy inhibitor such as SAR-405. In some embodiments, a protein kinase activator alone or in combination with a bromodomain inhibitor (e.g., ingenol-3,20-dibenzoate, JQ1, a pharmaceutically acceptable salt or derivative thereof, or a combination thereof) is administered in a combination therapy (i.e., co-administered) with: 1) one or more Bcl-2 inhibitor such as ABT-263 or MCL-1 inhibitor, and 2) an autophagy inhibitor such as SAR-405 or chloroquine. The therapeutic agents can be administered simultaneously, in sequence, or a combination thereof. For example, IDB, ABT-263, and SAR405 or chloroquine can be administered to a patient in need simultaneously. In other examples, at least two therapeutic agents (e.g., IDB and ABT-263) can be administered together to a patient in need followed by administering SAR405 or chloroquine. In further examples, IDB, ABT-263, and SAR405 or chloroquine can be administered at separate times to a patient in need. Other combinations of an effective amount of a protein kinase activator and optionally a bromodomain inhibitor with one or more anti-HIV agents, and one or more therapeutic agents that facilitate cell death are contemplated. As described herein, the therapeutic agents to inhibit HIV infection can be administered in separate formulations. Alternately, the therapeutic agents to inhibit HIV infection can be co-formulated, and the co-formulation administered to a subject.

Any of a variety of methods can be used to determine whether a treatment method is effective. For example, methods of determining whether the methods are effective in reducing immunodeficiency virus (e.g., HIV) viral load, and/or treating an immunodeficiency virus (e.g., HIV) infection, are any known test for indicia of immunodeficiency virus (e g , HIV) infection, including, but not limited to, measuring viral load, e.g., by measuring the amount of immunodeficiency virus (e.g., HIV) in a biological sample, e.g., using a polymerase chain reaction (PCR) with primers specific for an immunodeficiency virus (e.g.,

HIV) polynucleotide sequence; detecting and/or measuring a polypeptide encoded by an immunodeficiency virus (e.g., HIV), e.g., p24, gp120, reverse transcriptase, using, e.g., an immunological assay such as an enzyme-linked immunosorbent assay (ELISA) with an antibody specific for the polypeptide; and measuring the CD4⁺ T cell count in the individual.

The methods disclosed herein for treating or inhibiting human immunodeficiency virus (HIV) in a subject can kill 95% or greater (for e.g., 97% or greater, 99% or greater, or up to 100%) of infected cells within 2 days. Advantageously, uninfected cells are substantially resistant to apoptosis using the methods disclosed herein. In particular, 90% or greater (for e.g., 92% or greater, 94% or greater, 95% or greater, 97% or greater, 97% or greater, 99% or greater, or up to 100%) of uninfected cells are resistant to apoptosis using the methods disclosed herein.

Administration

The disclosed therapeutic agents can be administered either sequentially or simultaneously in separate or combined pharmaceutical formulations. When one or more of the disclosed therapeutic agents are used in combination with a second therapeutic agent the dose of each therapeutic agent can be either the same as or differ from that when the therapeutic agent is used alone. Appropriate doses will be readily appreciated by those skilled in the art.

The term “administration” and variants thereof (e.g., “administering” a therapeutic agent) in reference to a therapeutic agent of the invention means introducing the therapeutic agent or a prodrug of the therapeutic agent into the system of the animal in need of treatment. When a therapeutic agent of the invention or prodrug thereof is provided in combination with one or more other active agents (e.g., a cytotoxic agent, etc.), “administration” and its variants are each understood to include concurrent and sequential introduction of the therapeutic agent or prodrug thereof and other agents.

In vivo application of the disclosed therapeutic agents, and compositions containing them, can be accomplished by any suitable method and technique presently or prospectively known to those skilled in the art. For example, the disclosed therapeutic agents can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed therapeutic agents or compositions can be a single administration, or at continuous or distinct intervals as can be readily determined by a person skilled in the art.

The therapeutic agents disclosed herein, and compositions comprising them, can also be administered utilizing liposome technology, slow release capsules, implantable pumps, and biodegradable containers. These delivery methods can, advantageously, provide a uniform dosage over an extended period of time. The therapeutic agents can also be administered in their salt derivative forms or crystalline forms.

The therapeutic agents disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the therapeutic agents disclosed herein can be formulated such that an effective amount of the therapeutic agent is combined with a suitable carrier in order to facilitate effective administration of the therapeutic agent. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the therapeutic agents include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject therapeutic agents based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Therapeutic agents disclosed herein, and compositions comprising them, can be delivered to a cell either through direct contact with the cell or via a carrier means. Carrier means for delivering therapeutic agents and compositions to cells are known in the art and include, for example, encapsulating the composition in a liposome moiety. Another means for delivery of therapeutic agents and compositions disclosed herein to a cell comprises attaching the therapeutic agents to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Therapeutic agents can also be incorporated into polymers, examples of which include poly (D-L lactide-co-glycolide) polymer; poly[bis(p-carboxyphenoxy) propane:sebacic acid] in a 20:80 molar ratio (as used in GLIADEL); chondroitin; chitin; and chitosan.

For the treatment of infections, the therapeutic agents disclosed herein can be administered to a patient in need of treatment in combination with other antiviral substances to treat the infection. These other substances or treatments can be given at the same as or at different times from the therapeutic agents disclosed herein.

Therapeutic application of therapeutic agents and/or compositions containing them can be accomplished by any suitable therapeutic method and technique presently or prospectively known to those skilled in the art. Further, therapeutic agents and compositions disclosed herein have use as starting materials or intermediates for the preparation of other useful therapeutic agents and compositions.

Therapeutic agents and compositions disclosed herein can be locally administered at one or more anatomical sites, optionally in combination with a pharmaceutically acceptable carrier such as an inert diluent. Therapeutic agents and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active therapeutic agent can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active therapeutic agent, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active therapeutic agent can be incorporated into sustained-release preparations and devices.

Therapeutic agents and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin. Sterile injectable solutions are prepared by incorporating a therapeutic agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, therapeutic agents disclosed herein can be applied in as a liquid or solid. However, it will generally be desirable to administer them topically to the skin as compositions, in combination with a dermatologically acceptable carrier, which can be a solid or a liquid. Therapeutic agents and compositions disclosed herein can be applied topically to a subject's skin to treat an infection site. Therapeutic agents disclosed herein can be applied directly to the infection site. Preferably, the therapeutic agents are applied to the infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the therapeutic agents can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a therapeutic agent to the skin are disclosed in U.S. Patent No. 4,608,392; U.S. Pat. Nos. 4,992,478; 4,559,157; and 4,820,508.

Useful dosages of the therapeutic agents and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a therapeutic agent disclosed herein in combination with a pharmaceutically acceptable carrier. Pharmaceutical compositions adapted for oral, topical or parenteral administration, comprising an amount of a therapeutic agent constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The frequency of administration of a composition containing the therapeutic agent for reactivating latent HIV (such as protease inhibitor) can be any frequency that increases activation of the latent HIV infected cells to produce new HIV virus. Similarly, the frequency of administration of a composition containing the therapeutic agent for inhibiting HIV infection (such as an antiretroviral agent) can be any frequency that increases inhibition of HIV. The frequency of administration of a composition containing the therapeutic agent for eliminating or reducing infected HIV cell (such as Bcl 2 inhibitors and autophagy inhibitors) can be any frequency that increases the susceptibility of latently HIV infected cells to cell death upon HIV reactivation, thereby causing the latently HIV infected cells to die, without producing significant toxicity to the human. For example, the frequency of administration can be from about daily to about once a week. The frequency of administration can remain constant or can be variable during the duration of treatment. As with the effective amount, various factors can influence the actual frequency of administration used for a particular application. For example, the effective amount, duration of treatment, use of multiple treatment agents, route of administration, and severity of the HIV infection may require an increase or decrease in administration frequency.

In general, a single dose of an active agent is administered. In other embodiments, multiple doses of an active agent are administered. Where multiple doses are administered over a period of time, an active agent is administered, e.g., twice daily (qid), daily (qd), every other day (qod), every third day, three times per week (tiw), or twice per week (biw) over a period of time. For example, an active agent is administered qid, qd, qod, tiw, or biw over a period of from one day to about 2 years or more. For example, an active agent can be administered at any of the aforementioned frequencies for one week, two weeks, one month, two months, six months, one year, or two years, or more, depending on various factors. In some examples, the frequency of administration of the method steps disclosed herein can be at least once every 2-7 days, once every 2-3 days, such as once every 2 days (every other day).

Where two different active agents are administered, a first active agent and a second active agent can be administered in separate formulations. A first active agent and a second active agent can be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

An effective duration for administering the compositions, without producing significant toxicity to the human, can vary from several weeks to several months. In general, the effective duration for the treatment of an HIV infection as described herein can range in duration from at least 6 weeks, at least 7 weeks, or at least 8 weeks, from about 6 weeks to about 5 years, from about 6 weeks to about 1 year, from about 6 weeks to about 40 weeks, or from or about 6 weeks to about 5 months. Multiple factors can influence the actual effective duration used for a particular treatment. For example, an effective duration can vary with the frequency of administration, effective amount, use of multiple treatment agents, route of administration, and severity of the HIV infection being treated.

An active agent is administered to an individual using any available method and route suitable for drug delivery, including in vivo and ex vivo methods, as well as systemic and localized routes of administration. Conventional and pharmaceutically acceptable routes of administration include intranasal, intramuscular, intratracheal, transdermal, subcutaneous, intradermal, topical application, intravenous, vaginal, nasal, and other parenteral routes of administration. In some embodiments, an active agent is administered via an intravaginal route of administration. In other embodiments, an active agent is administered via an intrarectal route of administration. Routes of administration may be combined, if desired, or adjusted depending upon the agent and/or the desired effect. The composition can be administered in a single dose or in multiple doses. An active agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes. Parenteral routes of administration other than inhalation administration include, but are not necessarily limited to, topical, vaginal, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

An active agent can also be delivered to the subject by enteral administration. Enteral routes of administration include, but are not necessarily limited to, oral and rectal (e.g., using a suppository) delivery.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class Mammalia, and primates (e.g., humans, chimpanzees, and monkeys), that are susceptible to immunodeficiency virus (e.g., HIV) infection. In many embodiments, the hosts will be humans.

Kits

Kits for practicing the methods of the invention are further provided. By “kit” is intended any manufacture (e.g., a package or a container) comprising at least one reagent, e.g., anyone of the therapeutic agents described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the therapeutic agents based on the weight of the total composition including carrier or diluents. Although the dosage used will vary depending on the clinical goals to be achieved, a suitable dosage range of an active agent is one which provides up to about 1 mg to about 5000 mg, e.g., from about 1 mg to about 100 mg, from about 1 mg to about 50 mg, from about 1 mg to about 25 mg, from about 5 mg to about 200 mg, from about 5 mg to about 100 mg, from about 50 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects. Preferred dosages for a given compound are readily determinable by those of skill in the art by a variety of means. In some embodiments, an illustratively, dosage levels of the administered active ingredients can be: orally, 0.01 to about 100 mg/kg; intravenous, 0.01 to about 100 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

In some embodiments, the kit can include an effective amount of a therapeutic agent for reactivating latent HIV integrated into the genome of a cell infected with HIV in the subject, an effective amount of a therapeutic agent to inhibit HIV infection, and an effective amount of a therapeutic agent to eliminate or reduce the number of cells containing replication-competent HIV. For example, the kit can include a first component comprising ingenol-3,20-dibenzoate alone or in combination with a bromodomain inhibitor such as JQ 1 ; a second component comprising BMS-626529 (or its prodrug BMS-663068), raltegravir, or a combination thereof and a third component comprising navitoclax (ABT-263), an MCL-1 inhibitor such as AZD5991, 563845, or AMG 176; SAR-405, or a combination thereof.

In some embodiments, a suitable dose of ingenol-3,20-dibenzoate or a pharmaceutically acceptable salt, ester, or derivative thereof, is in the range of from about 0.1 mg/kg body weight to about 500 mg/kg body weight, e.g., from about 0.2 mg/kg body weight to about 500 mg/kg body weight, from about 0.1 mg/kg body weight to about 250 mg/kg body weight, from about 0.1 mg/kg body weight to about 100 mg/kg body weight, from about 0.1 mg/kg body weight to about 50 mg/kg body weight, from about 0.1 mg/kg body weight to about 25 mg/kg body weight, from about 0.1 mg/kg body weight to about 10 mg/kg body weight, from about 0.1 mg/kg body weight to about 5 mg/kg body weight, from about 0.1 mg/kg body weight to about 1.5 mg/kg body weight, from about 0.2 mg/kg body weight to about 250 mg/kg body weight, from about 0.2 mg/kg body weight to about 100 mg/kg body weight, from about 0.2 mg/kg body weight to about 50 mg/kg body weight, from about 0.5 mg/kg body weight to about 100 mg/kg body weight, from about 0.5 mg/kg body weight to about 50 mg/kg body weight, from about 0.5 mg/kg body weight to about 100 mg/kg body weight, or from about 0.5 mg/kg body weight to about 5 mg/kg body weight.

In some embodiments, a suitable dose of the bromodomain inhibitor such as JQ1 or a pharmaceutically acceptable salt, ester, or derivative thereof, is in the range of from about 1 mg/kg body weight to about 100 mg/kg body weight, e.g., from about 1 mg/kg body weight to about 90 mg/kg body weight, from about 1 mg/kg body weight to about 80 mg/kg body lo weight, from about 1 mg/kg body weight to about 70 mg/kg body weight, from about 1 mg/kg body weight to about 50 mg/kg body weight, from about 2 mg/kg body weight to about 100 mg/kg body weight, from about 5 mg/kg body weight to about 100 mg/kg body weight, from about 10 mg/kg body weight to about 100 mg/kg body weight, from about 2 mg/kg body weight to about 50 mg/kg body weight, from about 2 mg/kg body weight to about 30 mg/kg body weight, from about 2 mg/kg body weight to about 20 mg/kg body weight, from about 5 mg/kg body weight to about 50 mg/kg body weight, from about 5 mg/kg body weight to about 40 mg/kg body weight, from about 5 mg/kg body weight to about 30 mg/kg body weight, from about 10 mg/kg body weight to about 100 mg/kg body weight, or from about 10 mg/kg body weight to about 50 mg/kg body weight.

In some embodiments, a suitable dose of BMS-626529 (or BMS-663068) or a pharmaceutically acceptable salt, ester, or derivative thereof, is in the range of from about 1 mg/kg body weight to about 500 mg/kg body weight, e.g., from about 1 mg/kg body weight to about 500 mg/kg body weight, from about 2 mg/kg body weight to about 500 mg/kg body weight, from about 2.5 mg/kg body weight to about 500 mg/kg body weight, from about 3 mg/kg body weight to about 500 mg/kg body weight, from about 4 mg/kg body weight to about 500 mg/kg body weight, from about 5 mg/kg body weight to about 500 mg/kg body weight, from about 5 mg/kg body weight to about 100 mg/kg body weight, from about 5 mg/kg body weight to about 50 mg/kg body weight, from about 5 mg/kg body weight to about 20 mg/kg body weight, from about 1 mg/kg body weight to about 100 mg/kg body weight, from about 1 mg/kg body weight to about 50 mg/kg body weight, from about 2 mg/kg body weight to about 50 mg/kg body weight, from about 2 mg/kg body weight to about 30 mg/kg body weight, or from about 1 mg/kg body weight to about 25 mg/kg body weight.

In some embodiments, a suitable dose of raltegravir or a pharmaceutically acceptable salt, ester, or derivative thereof, is in the range of from about 1 mg/kg body weight to about 500 mg/kg body weight, e.g., from about 1 mg/kg body weight to about 500 mg/kg body weight, from about 2 mg/kg body weight to about 500 mg/kg body weight, from about 2.5 mg/kg body weight to about 500 mg/kg body weight, from about 3 mg/kg body weight to about 500 mg/kg body weight, from about 4 mg/kg body weight to about 500 mg/kg body weight, from about 5 mg/kg body weight to about 500 mg/kg body weight, from about 5 mg/kg body weight to about 100 mg/kg body weight, from about 5 mg/kg body weight to about 50 mg/kg body weight, from about 5 mg/kg body weight to about 20 mg/kg body lo weight, from about 1 mg/kg body weight to about 100 mg/kg body weight, from about 1 mg/kg body weight to about 50 mg/kg body weight, from about 2 mg/kg body weight to about 50 mg/kg body weight, from about 2 mg/kg body weight to about 30 mg/kg body weight, or from about 1 mg/kg body weight to about 25 mg/kg body weight.

In some embodiments, a suitable dose of navitoclax or a pharmaceutically acceptable salt, ester, or derivative thereof, is in the range of from about 1 mg/kg body weight to about 500 mg/kg body weight, e.g., from about 1 mg/kg body weight to about 500 mg/kg body weight, from about 2 mg/kg body weight to about 500 mg/kg body weight, from about 2.5 mg/kg body weight to about 500 mg/kg body weight, from about 3 mg/kg body weight to about 500 mg/kg body weight, from about 4 mg/kg body weight to about 500 mg/kg body weight, from about 5 mg/kg body weight to about 500 mg/kg body weight, from about 5 mg/kg body weight to about 100 mg/kg body weight, from about 5 mg/kg body weight to about 50 mg/kg body weight, from about 5 mg/kg body weight to about 20 mg/kg body weight, from about 1 mg/kg body weight to about 100 mg/kg body weight, from about 1 mg/kg body weight to about 50 mg/kg body weight, from about 2 mg/kg body weight to about 50 mg/kg body weight, from about 2 mg/kg body weight to about 30 mg/kg body weight, or from about 1 mg/kg body weight to about 25 mg/kg body weight.

In some embodiments, a suitable dose of MCL-1 inhibitor or a pharmaceutically acceptable salt, ester, or derivative thereof, is in the range of from about 1 mg/kg body weight to about 500 mg/kg body weight, e.g., from about 1 mg/kg body weight to about 500 mg/kg body weight, from about 2 mg/kg body weight to about 500 mg/kg body weight, from about 2.5 mg/kg body weight to about 500 mg/kg body weight, from about 3 mg/kg body weight to about 500 mg/kg body weight, from about 4 mg/kg body weight to about 500 mg/kg body weight, from about 5 mg/kg body weight to about 500 mg/kg body weight, from about 5 mg/kg body weight to about 100 mg/kg body weight, from about 5 mg/kg body weight to about 50 mg/kg body weight, from about 5 mg/kg body weight to about 20 mg/kg body weight, from about 1 mg/kg body weight to about 100 mg/kg body weight, from about 1 mg/kg body weight to about 50 mg/kg body weight, from about 2 mg/kg body weight to about 50 mg/kg body weight, from about 2 mg/kg body weight to about 30 mg/kg body weight, or from about 1 mg/kg body weight to about 25 mg/kg body weight.

In some embodiments, a suitable dose of SAR-405 or a pharmaceutically acceptable salt, ester, or derivative thereof, is in the range of from about 1 mg/kg body weight to about 500 mg/kg body weight, e.g., from about 1 mg/kg body weight to about 500 mg/kg body weight, from about 2 mg/kg body weight to about 500 mg/kg body weight, from about 2.5 mg/kg body weight to about 500 mg/kg body weight, from about 3 mg/kg body weight to about 500 mg/kg body weight, from about 4 mg/kg body weight to about 500 mg/kg body weight, from about 5 mg/kg body weight to about 500 mg/kg body weight, from about 5 mg/kg body weight to about 100 mg/kg body weight, from about 5 mg/kg body weight to about 50 mg/kg body weight, from about 5 mg/kg body weight to about 20 mg/kg body weight, from about 1 mg/kg body weight to about 100 mg/kg body weight, from about 1 mg/kg body weight to about 50 mg/kg body weight, from about 2 mg/kg body weight to about 50 mg/kg body weight, from about 2 mg/kg body weight to about 30 mg/kg body weight, or from about 1 mg/kg body weight to about 25 mg/kg body weight.

Also disclosed are kits that comprise a composition comprising a therapeutic agent disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more antimicrobial agents, such as those agents described herein. In one embodiment, a kit includes instructions or packaging materials that describe how to administer a therapeutic agent or composition of the kit. Containers of the kit can be of any suitable material, e.g., glass, plastic, metal, etc., and of any suitable size, shape, or configuration. In one embodiment, a therapeutic agent and/or agent disclosed herein is provided in the kit as a solid, such as a tablet, pill, or powder form. In another embodiment, a therapeutic agent and/or agent disclosed herein is provided in the kit as a liquid or solution. In one embodiment, the kit comprises an ampoule or syringe containing a therapeutic agent and/or agent disclosed herein in liquid or solution form.

Kits with unit doses of the active agent, e.g. in oral, vaginal, rectal, transdermal, or injectable doses (e.g., for intramuscular, intravenous, or subcutaneous injection), are provided. In such kits, in addition to the containers containing the unit doses will be an informational package insert describing the use and attendant benefits of the drugs in treating an immunodeficiency virus (e.g., an HIV) infection. Suitable active agents and unit doses are those described herein above.

In some embodiments, an active agent is packaged for oral administration. The present disclosure provides a packaging unit comprising unit dosages of an active agent. For example, the packaging unit is in some embodiments a conventional blister pack or any other form that includes tablets, pills, and the like. The blister pack will contain the appropriate number of unit dosage forms, in a sealed blister pack with a cardboard, paperboard, foil, or plastic backing, and enclosed in a suitable cover. Each blister container may be numbered or otherwise labeled, e.g., starting with day 1.

In some embodiments, a subject delivery system comprises an injection device. Exemplary, non-limiting drug delivery devices include injections devices, such as pen injectors, and needle/syringe devices. In some embodiments, the disclosure provides an injection delivery device that is pre-loaded with a formulation. The injection devices can be disposable, or reusable and refillable.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Clearance of HIV Infection by Selective Elimination of Host Cells Capable of Producing HIV

The RNA genome of the human immunodeficiency virus (HIV) is reverse-transcribed into DNA and integrated into the host genome, resulting in latent infections that are difficult to clear. Here it is shown an approach to eradicate HIV infections by selective elimination of host cells harboring replication-competent HIV (SECH), which includes viral reactivation, induction of cell death, inhibition of autophagy and the blocking of new infections. Viral reactivation triggers cell death specifically in HIV-1-infected T cells, which is promoted by agents that induce apoptosis and inhibit autophagy. SECH treatments can clear HIV-1 in >50% mice reconstituted with a human immune system, as demonstrated by the lack of viral rebound after withdrawal of treatments, and by adoptive transfer of treated lymphocytes into uninfected humanized mice. Moreover, SECH clears HIV-1 in blood samples from HIV-1-infected patients. The results show a strategy to eradicate HIV infections by selectively eliminating host cells capable of producing HIV.

Results

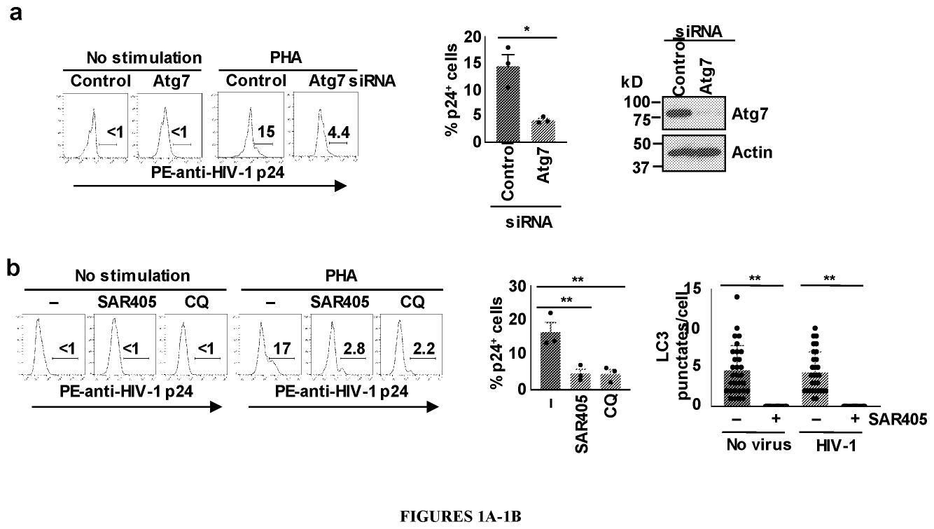

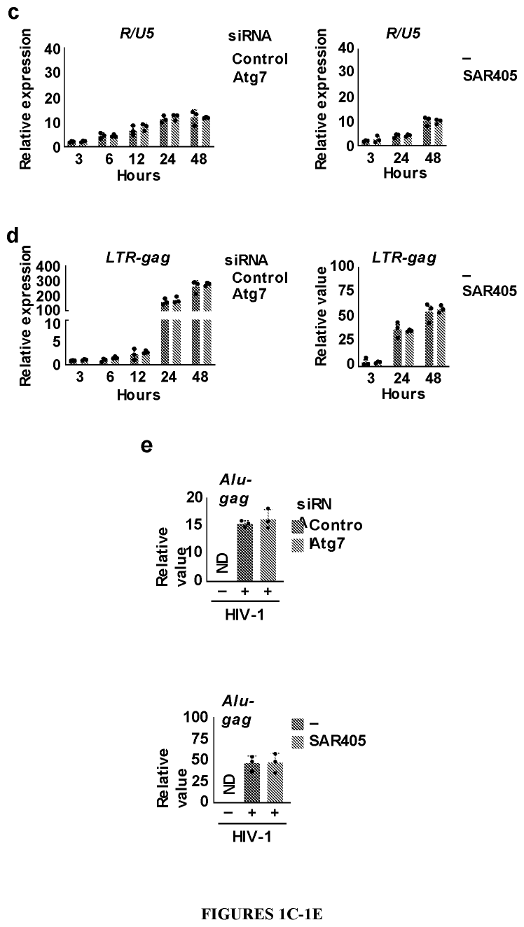

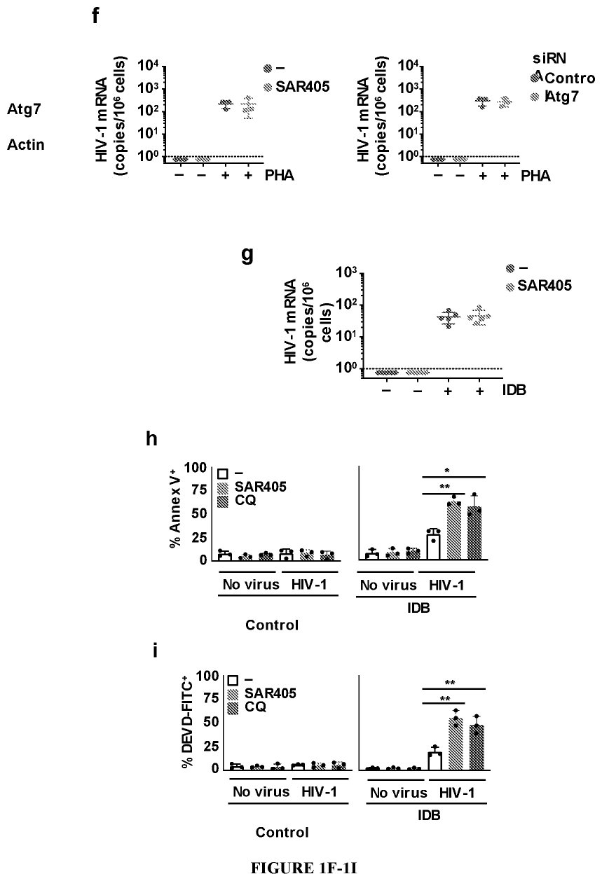

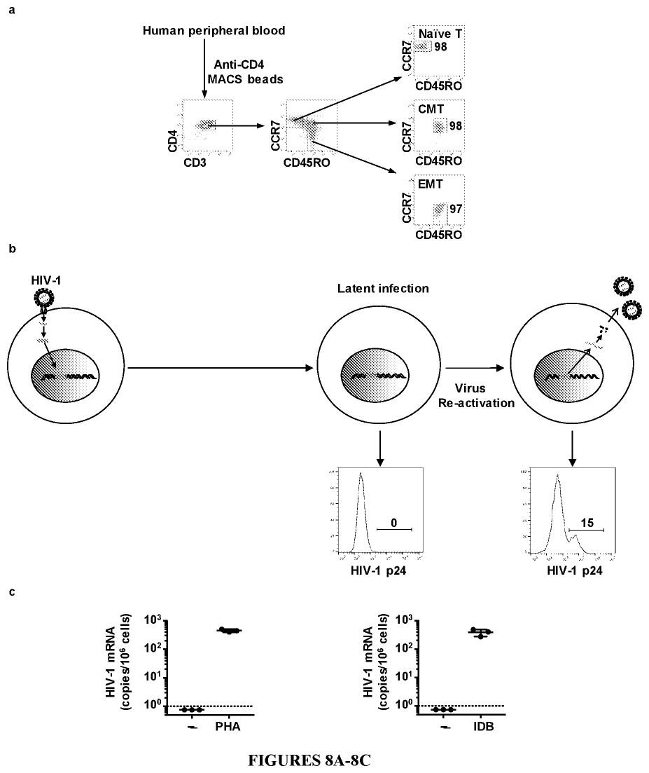

Inhibition of autophagy promotes host cell apoptosis. Since autophagy is important for the long-term protection of memory T cells, whether inhibition of autophagy can help to reduce the HIV-1 reservoir in these cells were first tested. CD3⁺CD4⁺CD45RO⁺CCR7⁺ central memory T cells were used (CMT; FIG. 8A) for HIV-1 infection and culture in the presence of CCL19 to establish HIV latent infection. Four days after infection with CXCR4-tropic HIV-1 NL4-3 at 0.1 multiplicity of infection (MOI), CMT showed no detectable expression HIV-1 Gag p24 protein (FIG. 1a, FIG. 8B). Stimulation with phytohemagglutinin (PHA) led to latency reversal of HIV-1 as shown by the induction of p24 expression (FIG. 1a, FIG. 8B). Latency reversal was also observed by the expression of HIV-1 mRNA after stimulation with PHA or ingenol 3,20-dibenzoate (IDB), a non-tumorigenic protein kinase c-ε activator (FIG. 8C). Interestingly, inhibition of autophagy in T cells by silencing the expression of an essential autophagy gene, Atg7, reduced the number of HIV-1 p24⁺ cells after PHA stimulation (FIG. 1a ). These data indicate that inhibition of autophagy can reduce the number of host T cells capable of producing HIV-1 p24.

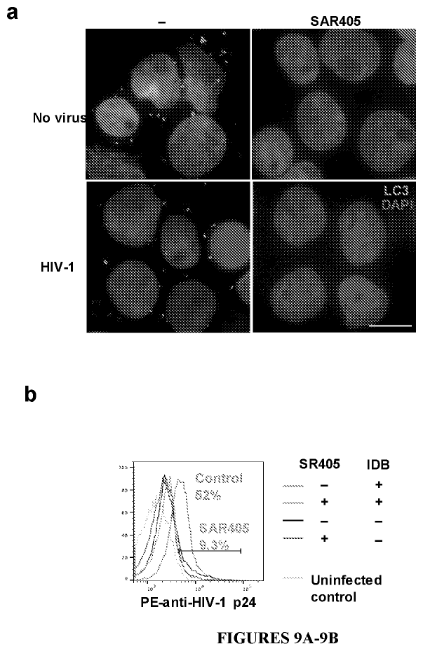

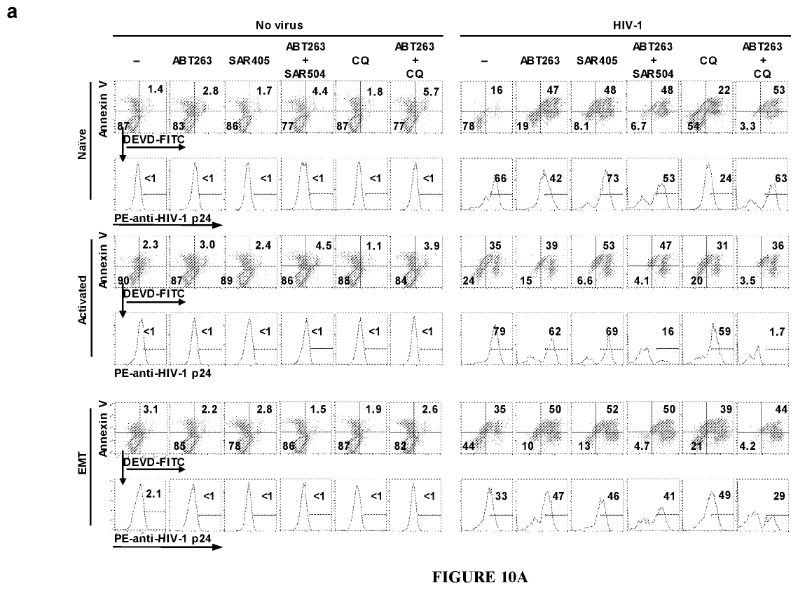

Consistent with the above findings, SAR405, an autophagy inhibitor that prevents autophagy initiation by suppressing VPS34, decreased the number of HIV-1 p24-producing T cells after latency reversal (FIG. 1b, FIG. 9A). Chloroquine (CQ), another autophagy inhibitor that blocks the progression of autophagolysomes, also reduced the number of HIV-1 p24-producing T cells after latency reversal (FIG. 1b). Also established was HIV latency in CMT infected with CCRS-utilizing HIV-1 AD8 at 1 MOI, and induced latency reversal with IDB (FIG. 8C). Induction of HIV-1 p24 expression by IDB-induced latency reversal was also inhibited by SAR405 (FIG. 9B). These data show that inhibition of autophagy can reduce the numbers of cells capable of producing HIV-1.

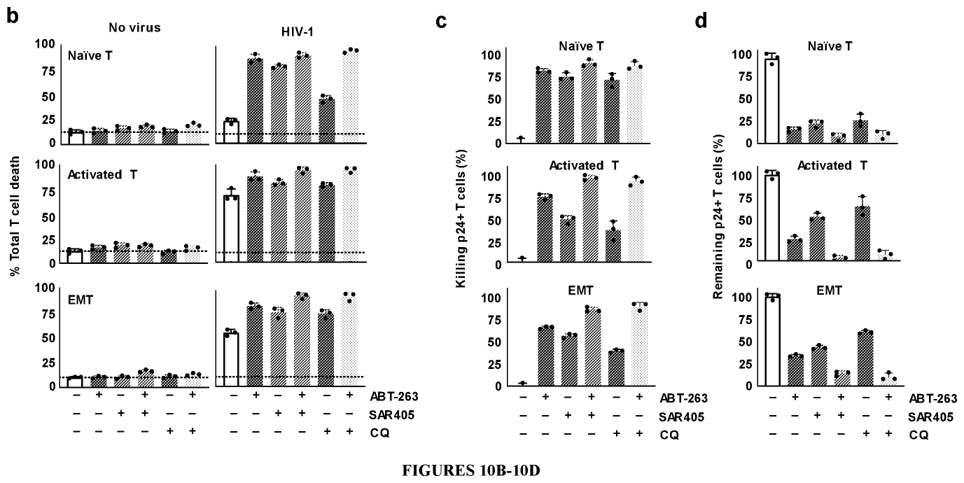

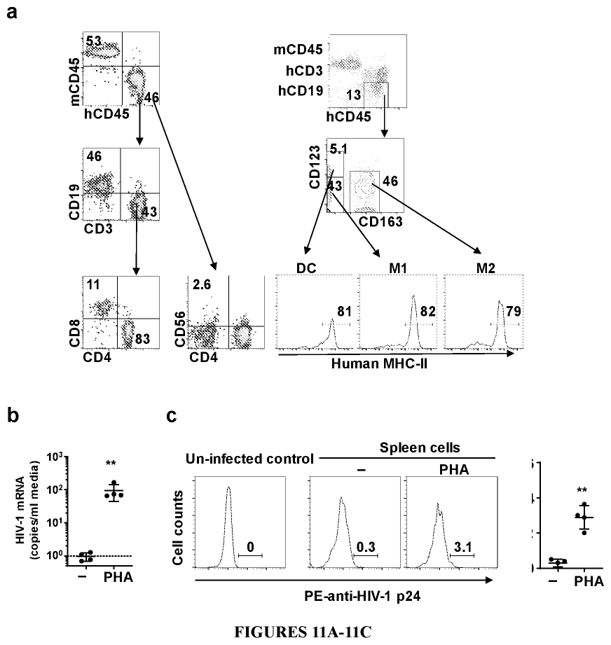

Inhibition of autophagy may reduce the number of HIV-1 p24⁺ cells after latency reversal by affecting different mechanisms, such as HIV-1 reverse transcription, integration of viral DNA into the host genome, viral reactivation and host cell survival. To distinguish between these possibilities, early and late products of reverse transcription by R/U5 and LTR-gag RT-PCR, respectively were measured. It was observed that the production of early and late HIV-1 transcripts, which indicates the level of reverse transcription, was not affected by silencing of Atg7 or treatment with SAR405 (FIGS. 1c,d). Next, integration of HIV-1 into the host genome by Alu-gag PCR were measured. It was found that inhibition of autophagy did not affect HIV-1 integration into the host genome (FIG. 1e). Induction of HIV-1 mRNA expression from latently infected cells was also unaffected by inhibition of autophagy (FIG. 10. Moreover, induction of HIV-1 mRNA expression in latently infected peripheral blood mononuclear cells (PBMCs) from ART-treated HIV-1 patients was not changed by SAR405 (FIG. 1g), indicating that autophagy is not required for the reactivation of latently infected HIV-1. Together, these data show that inhibition of autophagy does not have a direct effect on reverse transcription, integration and reactivation of latent HIV-1.

Next, it was determined whether inhibition of autophagy induced cell death in HIV-1-infected cells. It was observed that latency reversal with IDB induced cell death in HIV-1-infected CMT, as shown by Annexin V staining (FIG. 1h), and increased caspase-3 activities measured by cleavage of DEVD (FIG. 1i). Moreover, IDB-induced cell death in HIV-1-infected CMT was promoted by autophagy inhibitors SAR405 and CQ (FIGS. 1h,i). These data show that inhibition of autophagy promotes host cell death during latency reversal.

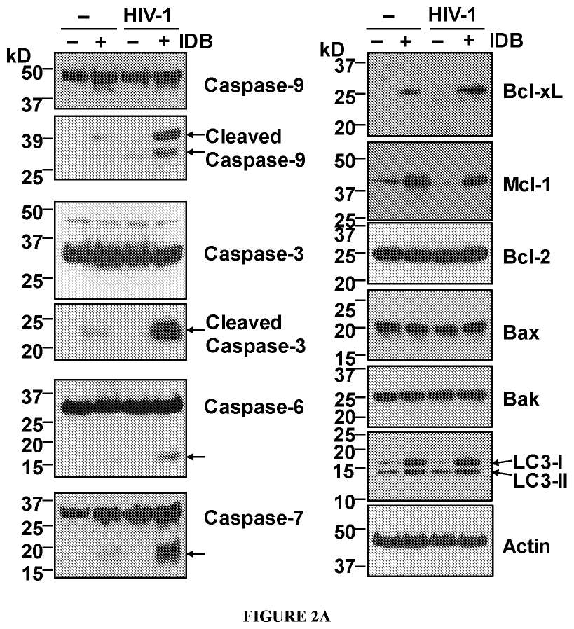

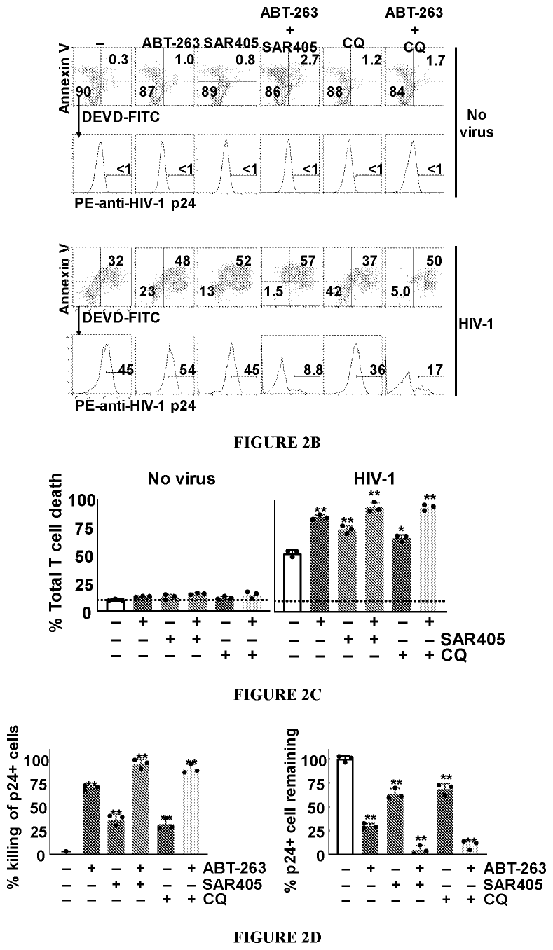

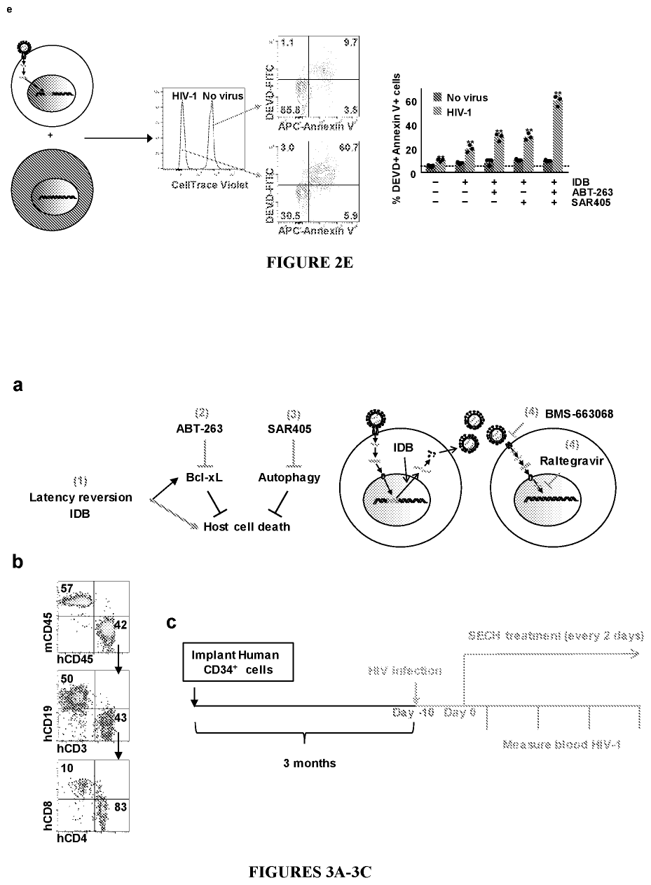

Specific killing of host cells by HIV-1 reactivation. It was found that IDB-mediated latency reversal induced the activation of caspase-9, caspase-3, caspase-6 and caspase-7 in HIV-1-infected T cells, as shown by the appearance of active processed forms of these caspases (FIG. 2a). The induction of caspase activation characteristic of apoptosis signaling is consistent with the possibility that latency reversal by IDB induces cell death in HIV-1-infected host cells (FIG. 1h,i). Treatment with IDB did not change the expression of anti-apoptotic Bcl-2, but increased the expression of anti-apoptotic Bcl-xL and Mc1-1 in CD4⁺ T cells not infected with HIV-1 (FIG. 2a). IDB also induced the expression of Bcl-xL and Mcl-1 in HIV-1-infected T cells (FIG. 2a). The increase in Bcl-xL expression in HIV-1-infected cells was greater than in uninfected controls (FIG. 2a). This indicates that HIV replication may synergize with IDB in inducing anti-apoptotic Bcl-xL, reminiscent of the roles for viral components in the regulation of Bcl-xL. Moreover, IDB also induced the expression of LC3 in T cell with or without HIV-1 infections (FIG. 2a), indicating that IDB promotes autophagy in T cells. While IDB can induce virus production in T cells harboring latent HIV-1 to trigger apoptosis, the up-regulation of anti-apoptotic Bcl-xL and Mc1-1 by IDB would counteract apoptosis signaling. The increases in anti-apoptotic molecules and autophagy in HIV-1-infected cells after latency reversal by IDB may explain in part why the use of latency reversal agents alone is not sufficient to clear HIV-1-infected cells. Nevertheless, the induction of anti-apoptotic molecules and autophagy by IDB may have the advantage by conferring resistance of uninfected T cells to the induction of cell death.

Killing of host cells by targeting apoptosis and autophagy. It was observed that virus reactivation by IDB induced cell death in HIV-1-infected T cells as shown by staining with Annexin V and cleavage of DEVD, while uninfected cells were relatively resistant (FIGS. 2b,c). Inhibition of autophagy with SAR405 increased IDB-induced killing of T cells latently infected by HIV-1 (FIG. 2b,c). Because latency reversal by IDB also showed the unintended effect of increasing anti-apoptotic molecules (FIG. 2a), targeting anti-apoptotic molecules in addition to reactivating the virus is potentially advantageous in promoting the killing of HIV-infected cells. Indeed, an inhibitor of Bcl-2 and Bcl-xL, ABT-263, increased IDB-mediated cell death in latently infected T cells, as shown by staining with Annexin V and DEVD-FITC (FIG. 2b,c). ABT-263 significantly increased the loss of viable HIV-1 p24⁺ cells in IDB-stimulated HIV-1-infected cells (FIG. 2d). This shows that counteracting anti-apoptotic molecules and inhibiting autophagy can promote the killing of HIV-1-infected T cells induced by latency reversal.

To eradicate HIV infections, all T cells that are capable of producing infectious HIV-1 need to be cleared. It was found that combining SAR405 with ABT-263 and IDB further increased cell death in HIV-infected T cells (FIG. 2b,c). Moreover, over 95% of HIV-1-p24⁺ cells in HIV-1-infected T cells could be killed within 2 days in vitro (FIG. 2d), while uninfected T cells were relatively resistant (FIG. 2b,c). Use of CQ instead of SAR405 also achieved similar results (FIG. 2b-d). These results show that latency reversal in combination with inhibition of autophagy and induction of apoptosis could efficiently kill HIV-1-infected T cells.

HIV-1 can infect and establish latency in both resting and activated T cells. In addition to memory T cells, certain levels of autophagy are present in T cells at all developmental stages. Therefore, whether inhibition of autophagy might affect the survival of HIV-1-infected CD4⁺ T cells with different differentiation and activation status in addition to CMT was examined, including CD3⁺CD4⁺CD45RO⁺CCR7⁻ effector memory T cells (EMT), CD3⁺CD4⁺CD45RO⁻CCR7⁺ naïve T cells (FIG. 8A), as well as CD4⁺ T cells activated with PHA and IL-2. It was found that the combination of IDB-induced latency reversal with inhibition of autophagy and promotion of apoptosis could kill a majority of these T cells infected by HIV-1 but not uninfected controls (FIG. 10A-10D). These results support the conclusion that the killing of HIV-1-infected T cells by latency reversal in combination with inhibition of autophagy and induction of apoptosis is selective.

Specific killing of HIV-1-infected cells but not bystanders. To further determine the selectivity in the killing of HIV-1-infected cells by latency reversal with inhibition of autophagy and promotion of apoptosis, uninfected T cells were labeled with CellTrace Violet and mixed them with T cells latently infected by HIV-1 (FIG. 2e). An attachment inhibitor that targets HIV-1 gp120 and blocks its binding to CD4⁺ T cells, BMS-626529, was added to prevent new rounds of infection. It was observed that treatments with IDB, ABT-263 and SAR4505 alone or together killed HIV-1-infected cells, while uninfected cells were resistant (FIG. 2e). These results show that IDB-induced latency reversal, in combination with the inhibition of autophagy and promotion of apoptosis, leads to selective killing of HIV-1-infected cells but not the uninfected bystanders.

Virus reactivation by IDB potentially confers the specificity in the killing of HIV-1-infected cells by selectively inducing caspase activation and apoptosis in cells harboring HIV-1 infections (FIG. 2a). The unintended effect of up-regulating anti-apoptotic molecules and autophagy by IDB may be mitigated by using ABT-263 and SAR405. Inhibition of autophagy could help to remove an important protective mechanism for cell survival, thereby unleashing the cell death pathways in HIV-1-infected T cells triggered by IDB and by pro-apoptotic ABT-263 (FIG. 2b,c; FIG. 10). IDB-induced upregulation of anti-apoptotic Bcl-xL and Mcl-1, as well as the induction of autophagy in cells not infected by HIV-1, may protect uninfected cells against cell death (FIG. 2a), thereby increasing the specificity in the killing of HIV-1-infected cells.

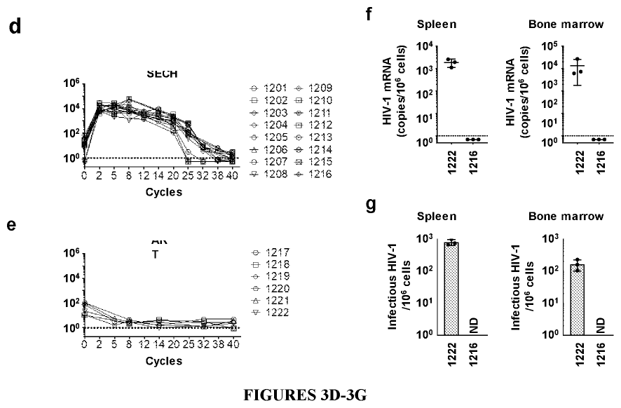

Clearance of HIV-1 infections in humanized mice by SECH. Next, it was determined whether it was possible to clear HIV-1 infection in vivo by killing HIV-1-infected cells. A humanized mouse model containing a reconstituted human immune system that had been established for HIV-1 infection and cure studies was used. A SECH approach was designed for selective elimination of host cells capable of producing HIV, including the use of IDB for latency reversal, in combination with agents that induce apoptosis and inhibit autophagy to eliminate HIV-1-infected cells in humanized mice (FIG. 3a). However, IDB is expected to induce the production of new HIV-1 virions that can infect other cells. Antiretroviral drugs that aim to inhibit the HIV-1 fusion and integration (FIG. 3a) were included. A prodrug was used for attachment inhibitor BMS-626529, BMS-663068, with increased solubility and can be converted to the active and cell permeable BMS-626529 by alkaline phosphatase in the intestine, as well as raltegravir, an integrase strand transfer inhibitor that prevents viral integration. Other drugs commonly used in cART, such as reverse transcriptase inhibitors and protease inhibitors, were not included. This would thus permit viral production to induce cell death signaling in infected cells while preventing virus spread, thereby protecting neighboring healthy cells from infection by newly produced HIV-1.

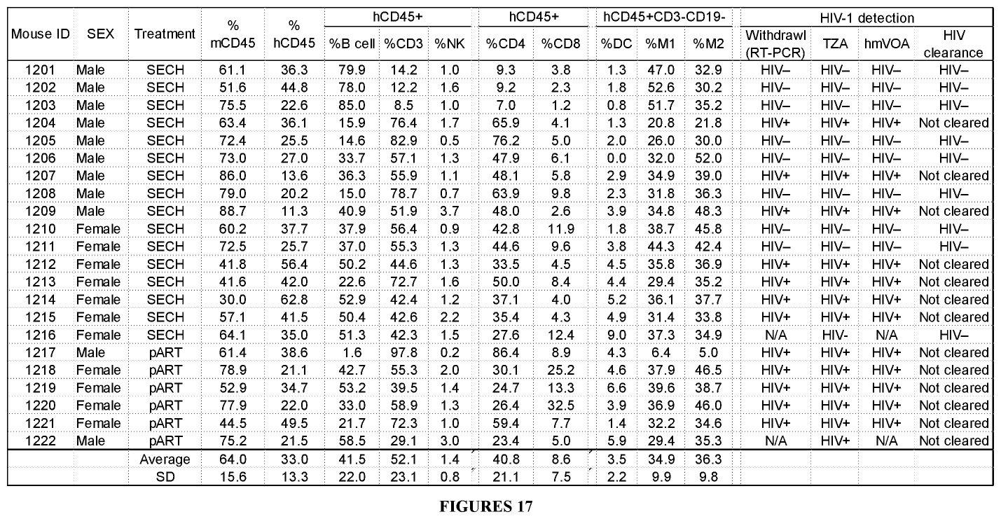

To generate mice implanted with human CD34⁺ hematopoietic stem cells (Hu-HSC mice), immunodeficient NSG-SGM3 mice were used with transgenic expression of human IL-3, GM-CSF and SCF that support the stable engraftment of a variety of cell types in the immune system, including both myeloid and lymphoid lineages. After engraftment of human CD34⁺ stem cells, mice were efficiently reconstituted with human CD4⁺ and CD8⁺ T cells, B cells, NK cells, dendritic cells and macrophages (FIG. 3b; FIG. 11a; FIG. 17). Hu-HSC mice used for this set of experiment were reconstituted with an average of 33% human CD45⁺ cells (FIG. 17). Among these human CD45⁺ cells, 40.8% were CD4⁺ T cells (FIG. 17). These mice were then infected with HIV-1 (FIG. 3c). Recent data suggest that the establishment of refractory viral reservoirs takes place rapidly in human HIV-1 patients and in SIV-infected rhesus monkeys. Some Hu-HSC mice were examined 10 days after HIV-1 infection. Stimulation of splenocytes with PHA induced the expression of HIV-1 mRNA and production of Gag p24 in CD4⁺ T cells (FIG. 11b,c). Although HIV-1 protein expression could still be detected in some T cells before the start of ART, these data show that latent HIV-1 infection has been established in human CD4⁺ T cells in these Hu-HSC mice.

Then the effects of SECH treatments in these mice in vivo were determined. SECH treatments contained IDB (2.5 mg/kg b.w.), ABT-263 (50 mg/kg b.w.) and SAR405 (50 mg/kg b.w.) formulated in a solvent mixture 10% ethanol, 30% polyethylene glycol 400 and 60% Phosal 50 PG (EPP) for delivery into mice by oral gavage. Pilot experiments with administration of these doses of drugs to wild type C57BL/7 mice or in NSG-SGM3-derived HSC-Hu mice daily for 2 weeks showed no loss of body weight or other adverse effects on mice. SECH treatment was started at day 10 post-infection once every two days as once cycles of treatments (FIG. 3c). Raltegravir and BMS-663068 (20 mg/kg b.w.) were included as the ART regimen daily. Mice in the control group received ART only daily.

HIV-1 mRNA were monitored in the mouse peripheral blood by RT-PCR. The purpose of ART included in the SECH protocol was to prevent the spread of HIV to uninfected cells, but not to decrease the production of new HIV-1 from induced cells induced by virus reactivation (FIG. 3a). A burst of new HIV-1 production induced by virus reactivation in the SECH group (FIG. 3d) was observed. A decline in HIV-1 mRNA detected in the blood would suggest a reduction in the HIV-producing cell pool. Indeed, it was found that HIV-1 in the peripheral blood decreased after 25 to 32 cycles of SECH treatments (FIG. 3d). Between 32 and 40 cycles of SECH treatments, most mice treated by SECH showed either reduced or undetectable HIV-1 in the peripheral blood (FIG. 3d). As expected, mice in the ART-treated group showed low or undetectable HIV-1 throughout the treatments (FIG. 3d).

Determination of HIV-1 clearance in Hu-HSC mice. To determine whether HIV-1-producing cells were cleared in Hu-HSC after 40 cycles of SECH treatments, HIV-1 was examined in the spleen and bone marrow from mouse 1216 treated by SECH and mouse 1222 treated by ART. Detect were HIV-1 mRNA in the spleen and bone marrow of mouse 1222 but not mouse 1216 (FIG. 30. TZM-bl cells stably expressing CD4, CCRS, CXCR4 and carrying a β-galactosidase gene under the control of HIV-1 long terminal repeat promoter have been used for more sensitive detection of replication-competent HIV-1 than traditional virus outgrowth assays or quantitative RT-PCR^{51,52}. By this highly sensitive TZA assay, infectious HIV-1 were detected in the spleen and bone marrow cells of mouse 1222 but not mouse 1216 (FIG. 3g). These results indicate that infectious HIV-1 is present in mouse 1222 treated by ART, but absent in mouse 1216 treated by SECH.

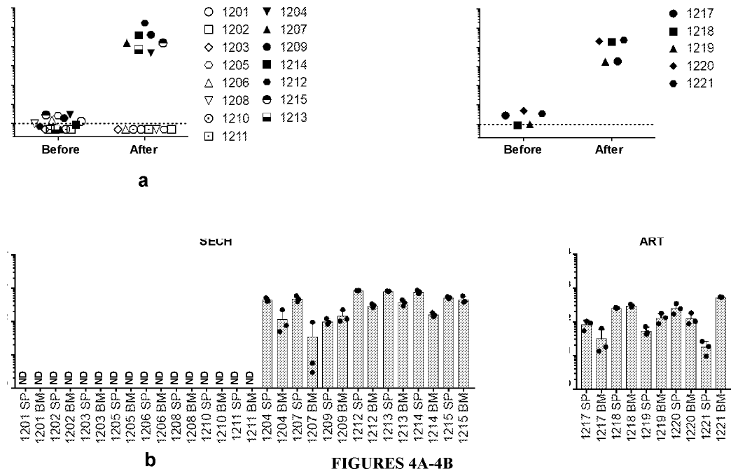

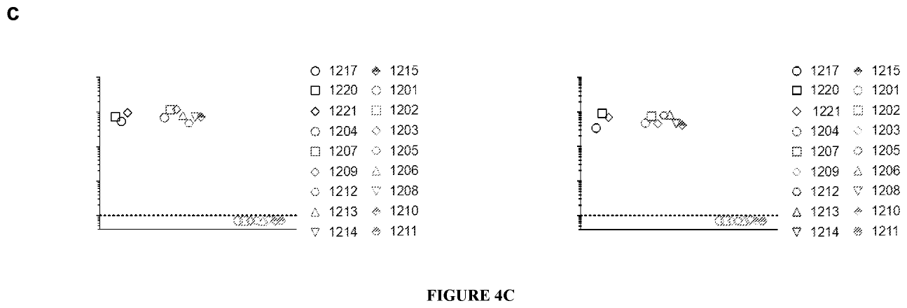

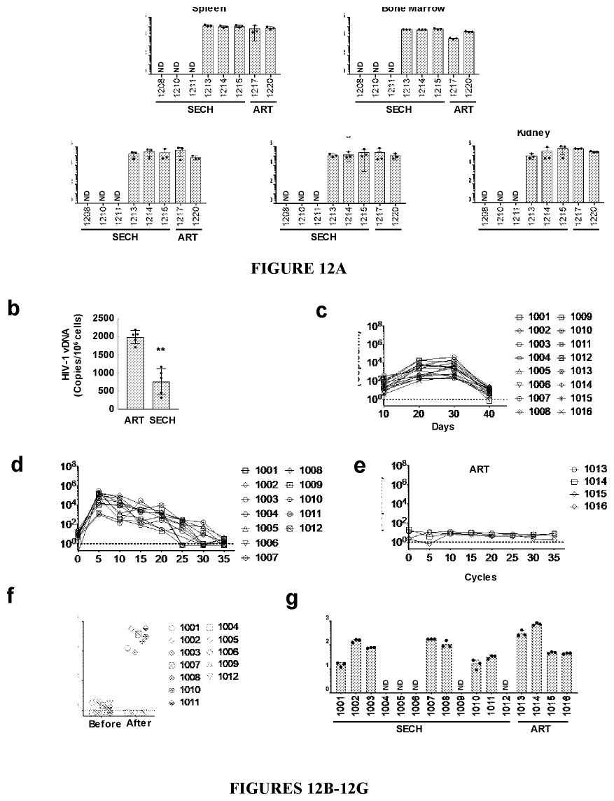

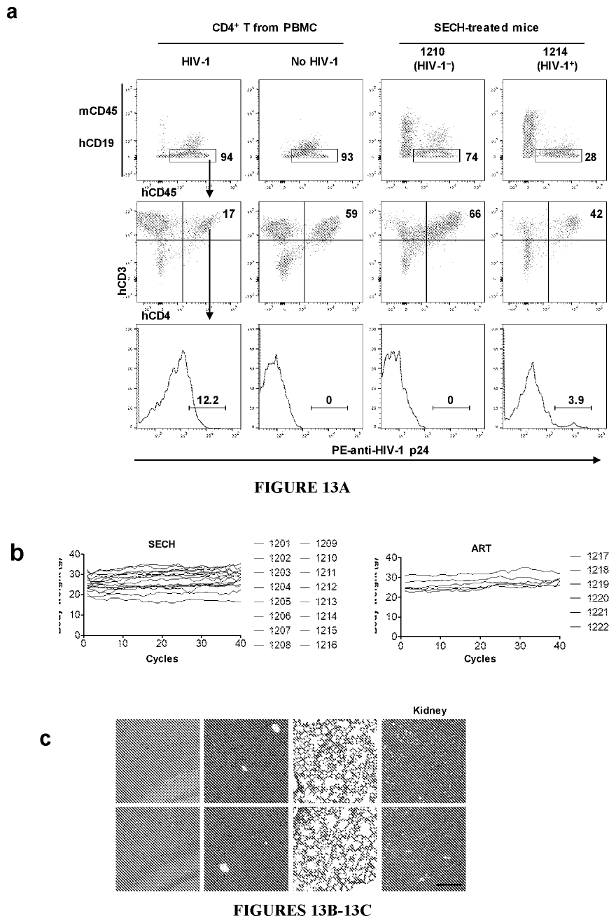

To further determine whether HIV-1 was cleared in Hu-HSC, SECH treatments were stopped after 40 cycles, followed by 2 months of withdrawal of treatments. Then measured was HIV-1 in the peripheral blood of these mice. Eight mice were found to be negative for HIV-1 in the blood, while seven mice showed HIV-1 rebound after withdrawal of SECH (FIG. 4a). It was found that those mice with no HIV-1 rebound in the blood also did not produce HIV-1 from spleen and bone marrow cells by the TZA assay (FIG. 4b). In contrast, all mice treated by ART showed virus rebound (FIG. 4a,b). In SECH-treated mice lacking detectable HIV-1 in the spleen and bone marrow, RT-PCR also showed no detectable HIV-1 in the lung, liver and kidney (FIG. 12a). Clearance of infectious HIV-1 is correlated with a decrease in HIV-1 DNA (FIG. 12b). Lower levels of viral DNA are consistent with the clearance of productive HIV-1. The remaining viral DNA in mice with the clearance of productive infection likely represents non-productive HIV infection. Consistent with this possibility, an HIV-1 patient treated by stem cell transplantation is free of the infectious virus but still contains residue HIV-1 DNA in the tissues. These observations support the conclusion that HIV-1 cure can be achieved by clearing replication-competent HIV-1 without removing the non-productive virus. Intracellular staining of T cells also showed the lack of p24 production in mice free of HIV-1 (FIG. 13a). In contrast, all mice with HIV-1 rebound showed HIV-1 production in spleen and bone marrow cells (FIG. 4b). All mice treated by

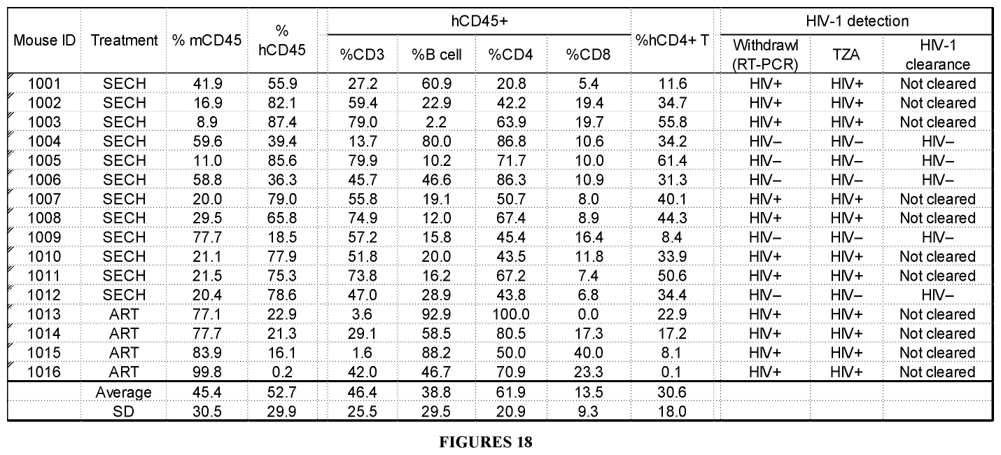

ART also remained HIV-1⁺(FIG. 4a,b). This indicates that HIV-1 was cleared from more than 50% of Hu-HSC mice treated by SECH. In contrast, ART did not clear HIV-1 although it could control active viral production. In parallel experiments, it was observed that HIV-1 infections in Hu-HSC mice could be suppressed by ART (FIG. 18, FIG. 12c). Further treatments by SECH could clear HIV-1 in a apportion of these mice, as shown by lack of virus rebound after withdrawal of the treatments, and by TZA assays (FIG. 12d-g). This suggests that SECH is potentially effective for treating HIV-1 infections with or without prior ART treatments.

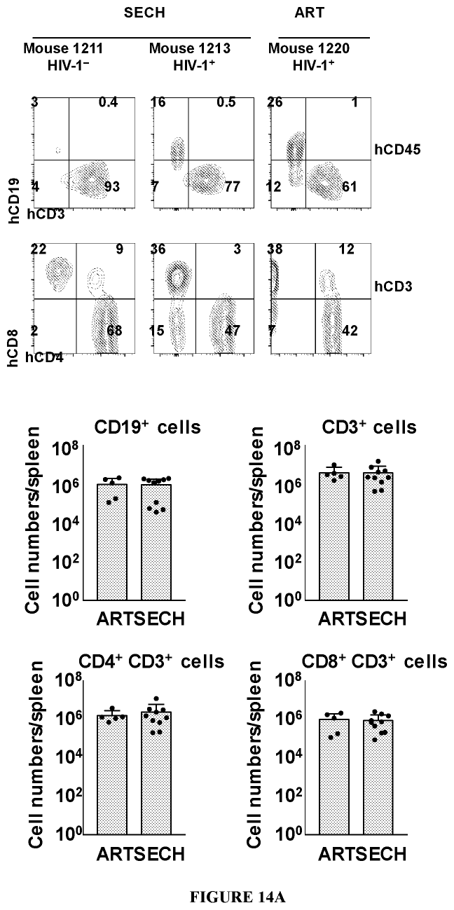

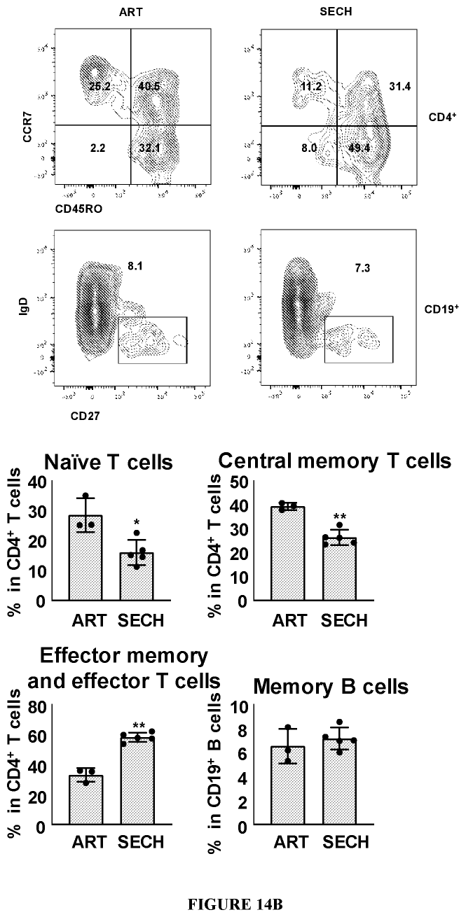

Validation of HIV clearance by an in vivo outgrowth assay. In an in vivo humanized mouse based-virus outgrowth assay (hmVOA) through adoptive transfer of HIV-1-infected cells into humanized mice, the preformed lymphoid organs in the recipients provides highly sensitive detection of latent HIV-1 infections^{14,55,56}. To further confirm virus clearance in the SECH-treated mice, spleen and bone marrow cells were transferred from these mice into uninfected Hu-HSC mice. Consistent with the in vitro TZA assay, HIV-1 was not detected by hmVOA in uninfected recipients after adoptive transfer of spleen or bone marrow cells from HIV-1-negative mice (FIG. 4c). In contrast, all Hu-HSC mice became HIV-1⁺after receiving spleen or bone marrow cells from HIV-1⁺mice (FIG. 4c). These data demonstrate that mice negative for HIV-1 by TZA assay were indeed free of virus by in vivo virus outgrowth assay in recipient Hu-HSC mice. These results suggest that the SECH treatments led to the elimination of HIV-1 infections in 8 out of 15 Hu-HSC mice as shown by the lack of virus rebound after withdrawal of SECH treatments, and the lack of infectious viruses by in vitro TZA assay and by in vivo hmVOA assay. No significant loss of body weight was observed in these mice after SECH or ART treatments (FIG. 13b). Also no signs of inflammation or other histological changes was observed in the brain, liver, lung and kidney of these mice after SECH treatments (FIG. 13c). SECH- and ART-treated mice showed overall comparable levels of human CD19⁺ B cells and CD4⁺ or CD8⁺ T cells (FIG. 14a). In SECH-treated mice, there were increases in CD45RO⁺CCR7⁻ effector memory T cells and CD45RO⁻ CCR7⁻ effector T cells, while CD45RO⁺CCR7⁺ central memory T cells and CD19⁺CD27⁺memory B cells still present (FIG. 14b). These results indicate that SECH is safe for treating HIV-1-infected Hu-HSC mice to clear HIV-1 infections.

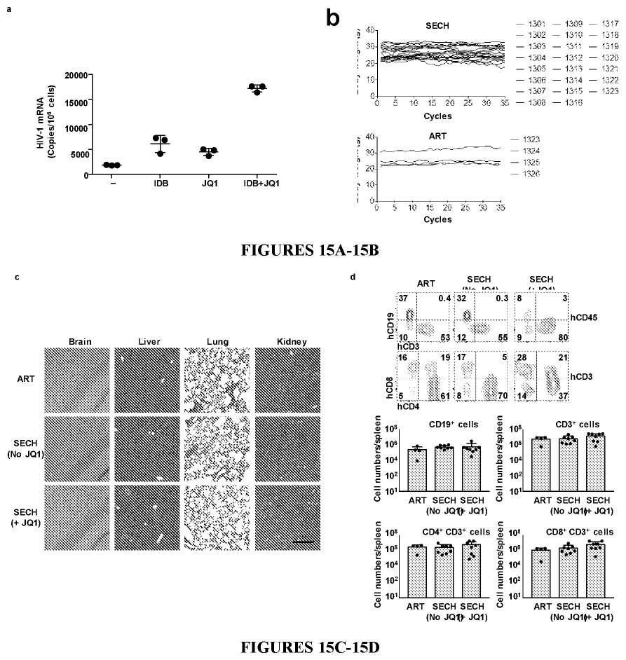

Synergy between JQ1 and IDB in HIV-1 clearance by SECH. Why HIV-1 is only cleared in a portion of the infected Hu-HSC mice is unclear. It is possible that improving HIV-1 reactivation induced by IDB could promote viral clearance. Bromodomain containing 4 (BRD4) is a negative regulator of transcription factor PTEF-b required for HIV-1 gene expression. JQ1, an inhibitor for the BET family of bromodomains, can promote the reactivation of HIV-1. Consistent previous observations, it was observed that using JQ1 and IDB together induced better reactivation of HIV-1 from latently infected T cells (FIG. 15a). Therefore, whether including the epigenetic modifier JQ1 could increase the efficacy of SECH treatment was investigated.

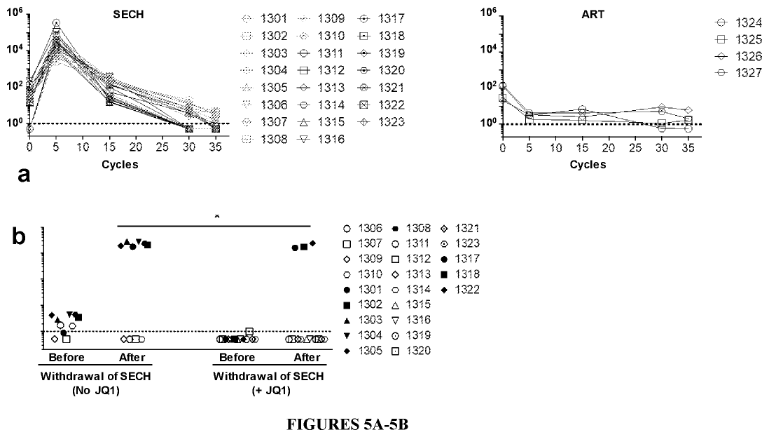

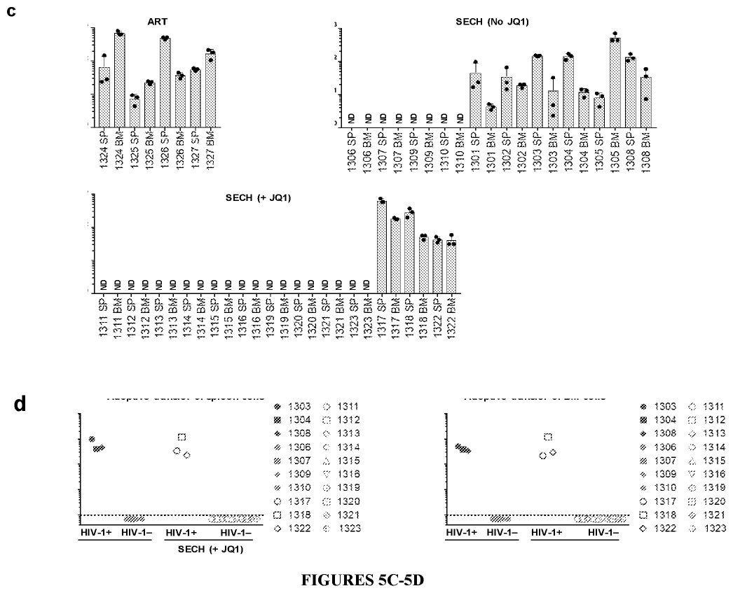

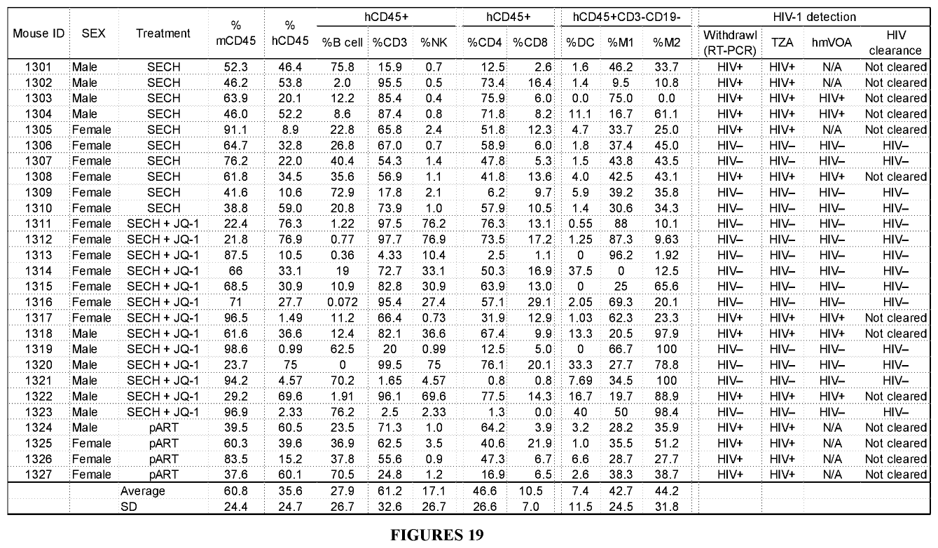

SECH treatments were compared with or without the inclusion of JQ1 in HIV-1-infected Hu-HSC mice. Hu-HSC mice were reconstituted with an average of 35.6% human CD45⁺cells (FIG. 19). Among human CD45⁺cells, 46.6% were CD4⁺T cells (FIG. 19). After the initial burst of HIV-1 mRNA induced by virus reactivation by SECH, all mice showed either low or undetectable HIV-1 in the peripheral blood after 35 cycles of SECH treatments (FIG. 5a). After 2 months of withdrawal of treatments, 4 out of 10 mice in the SECH group without JQ1 showed no HIV-1 rebound (FIG. 5b). Interestingly, 10 out of 13 mice showed no virus rebound when JQ1 was included in the SECH treatments (FIG. 5b). No HIV-1 production was detected in the spleen and bone marrow cells from these HIV-1-negative mice by TZA assay (FIG. 5c). Moreover, HIV-1 was not detected by hmVOA assays after adoptive transfer of spleen and bone marrow cells from these HIV-1-negative mice into Hu-HSC recipients (FIG. 5d). In contrast, HIV-1⁺ mice after SECH treatments and all ART-treated mice produced HIV-1 by in vitro TZA and in vivo hmVOA assays (FIG. 5c,d). Additionally, no significant differences in body weight, tissue sections and the total numbers of T or B cells were observed in these mice after treatment by SECH or ART (FIG. 15b-15d). These results suggest that inclusion of JQ1 together with IDB for latency reversal can enhance the efficacy of HIV-1 clearance by SECH.

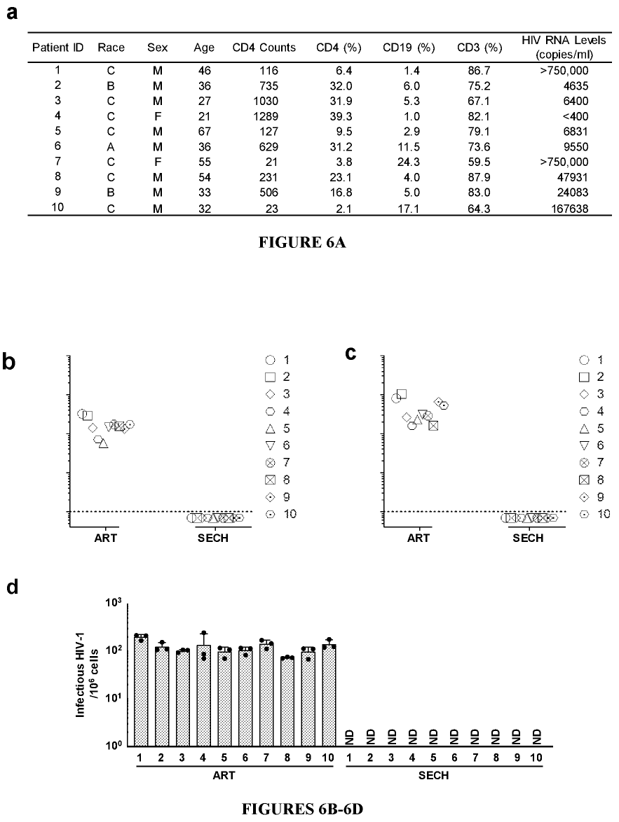

Clearance of HIV-1 in PBMCs of HIV-1-infected patients. It was next determined whether SECH could be used to clear HIV-1 infections in PBMCs from HIV-1-infected patients. PBMCs were examined from 10 ART-naïve HIV-1 patients who had not received previous antiretroviral treatments. Five of these patients have relatively normal CD4⁺T cell counts (>500411 blood), while four showed severely depletion of CD4⁺T cells (<200/μl; FIG. 6a). PBMCs from each patient were separated into two fractions for treatments by either SECH or ART, with a two-day culture as one cycle of treatments. After 5 cycles of treatments, HIV-1 was undetectable in PBMCs treated by SECH, but remained detectable after treatment by ART (FIG. 6b). Consistent with RT-PCR analyses, SECH-treated PBMCs did not produce HIV-1 after adoptive transfer into Hu-HSC recipient mice, while the same patient samples treated by ART produced the virus in the recipients (FIG. 6c). TZA assays also showed the lack of infectious HIV-1 in cells treated by SECH (FIG. 6d). This suggests that the SECH treatments successfully eliminated HIV-1 in the blood samples of HIV-1-infected patients.

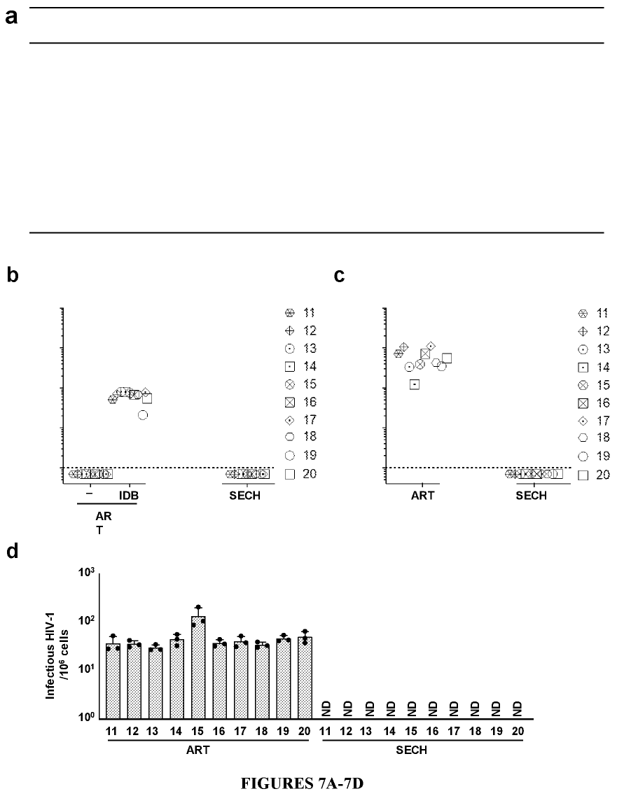

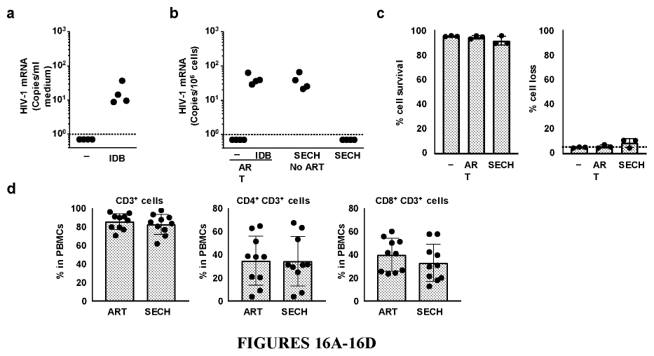

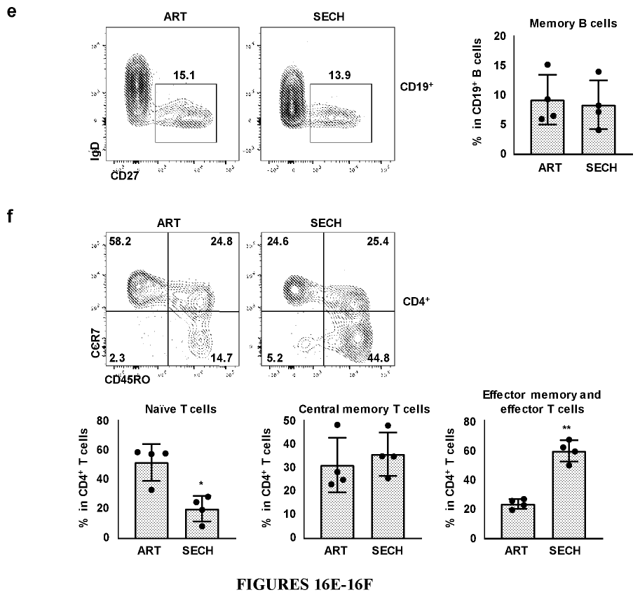

Also examined were PBMCs from outpatients undergoing ART treatments at the Houston Methodist Hospital (FIG. 7a). While PBMCs of these patients showed no active virus production, HIV-1 could be detected after stimulation with IDB (FIG. 7b, FIG. 16a), suggesting that these patients harbored latent HIV-1 infections. After five cycles of treatments by SECH, it was observed that HIV-1 was absent in PBMCs from these patients as shown by RT-PCR (FIG. 7b), indicating that SECH is effective for clearing HIV-1 in PBMCs from ART-treated patients. To confirm this finding, adoptive transfer of treated PBMCs into uninfected Hu-HSC mice for in vivo hmVOA assay were performed. It was found that SECH-treated PBMCs from these patients did not produce HIV-1, while all ART-treated samples were HIV-1⁺ by hmVOA (FIG. 7c). Consistently, TZA assays also showed that the cells treated by SECH did not produce infectious HIV-1 (FIG. 7d). SECH treatment without the inclusion of ART drugs did not completely clear HIV-1 (FIG. 16b), suggesting that blocking new viral infections is preferred for SECH. Similar to earlier observations for in vitro cultures (FIG. 2e), the cell viabilities and total live cells of patient PBMCs were similar between untreated controls and samples treated by SECH or ART (FIG. 16c). Interestingly, the levels of total T cells were comparable between samples treated by SECH and ART (FIG. 16d). Memory B cells were comparable in samples treated by SECH and ART (FIG. 16e). In SECH-treated samples, there were increases in CD45RO⁺CCR7⁻ effector memory T cells and CD45RO⁻CCR7⁻ effector T cells, while CD45RO⁺CCR7⁺ central memory T cells were not significantly changed (FIG. 160. This indicates that SECH treatments killed infected T cells but not uninfected cells. SECH therefore can potentially be developed into an effective strategy to treat for HIV-1 infections.

Discussion In this study, it was shown that it is feasible to clear HIV-1 infection by the SECH approach through selective elimination of host cells capable of producing virus. This method combines agents that reactivate HIV-1, promote cell death and inhibit autophagy, together with ART to prevent new infections. Continuous cycles of SECH treatments could clear HIV-1 infection in over 50% of Hu-HSC mice in vivo. HIV-1 clearance was confirmed by a lack of virus rebound after withdrawal of SECH treatments, in vitro TZA assays, and in vivo virus outgrowth assay by adoptive transfer of the spleen and bone marrow cells from treated mice into uninfected humanized mice. Treatment of PBMCs from HIV-1-infected patients by SECH agents also cleared HIV-1 in PBMCs as determined by humanized mouse-based virus outgrowth assay. SECH is potentially useful for clearing HIV-1 infections in Hu-HSC mice or in patient PBMCs with or without prior ART treatments. The example shows a strategy for the eradication of HIV-1 infection by selectively eliminating the infected cells that are capable of producing new viruses.

The SECH approach can induce the deletion of HIV-1-infected cells but not the uninfected healthy cells. Mechanistically, activation of T cells with a latency reversing agent, IDB, led to caspase activation and cell death signaling in HIV-infected cells, while sparing the uninfected cells (FIG. 2a, e). Applying IDB has an unintended effect of inducing Bcl-xL and Mcl-1, which likely inhibit the killing of HIV-1-infected cells (FIG. 2a). ABT-263 is used to mitigate this unintended effect of IDB by counteracting anti-apoptotic molecules to promote cell death in HIV-infected cells. Nevertheless, the induction of Bcl-xL and Mc1-1 by IDB may have the benefit of conferring protection of uninfected cells against killing, thereby improving the specificity of the SECH approach in targeting HIV-1-infected cells. An epigenetic modifier, JQ1, could be included in SECH to synergize with IDB for HIV-1 reactivation to promote the clearance of HIV-1-producing cells. Preferential killing of HIV-1-infected cells and protection of uninfected cells are important for the selectivity and safety of this SECH method, resulting in the eradication of HIV reservoir while preserving a normal immune cell repertoire.

Although the agents for SECH can cause rapid and specific clearance of HIV-1-infected cells in vitro (FIGS. 2e, 6 and 7), the efficacy in vivo is expected to be lower. Indeed, it was observed that clearance of HIV-1 reservoirs in different sets of HIV-1-infected Hu-HSC mice ranged from 40% to 70% (FIGS. 3-5 and FIG. 12). Whether the drugs for SECH can reach different tissues at sufficient amounts in vivo is not known. The pharmacokinetics for these drugs in vivo and the duration of effective drug concentrations present in different tissues remain to be characterized. This will help to determine the optimal doses and frequency of treatments. It was found that the success rate for SECH treatments can be improved by increasing the reactivation of latent HIV infection. Inclusion of epigenetic modifier JQ1 further increased HIV-1 reactivation and clearance (FIG. 5, FIG. 15a), suggesting that increased virus reactivation can improve the success rate of viral clearance by SECH. Therefore, promoting virus reactivation represents one effective way for improving the success rate of SECH in HIV-1 clearance in vivo. Testing alternative latency reversal drugs may reveal better approaches for virus reactivation to facilitate the clearance of HIV-1 reservoirs.

Autophagy is essential for the protection of long-term maintenance of memory T cells and memory B cells, and promoting the longevity of other cell types as well. It has been lo shown that autophagy can regulate HIV-1 replication during persistent infection. HIV-1 exploits the properties of longevity and quiescence of memory T cells to establish latent infections. It was found that autophagy did not affect HIV-1 reverse transcription, integration into host genome or reactivation. Rather, inhibition of autophagy promoted caspase activation and cell death induced by virus reaction with IDB (FIG. 1h,i and FIG. 2b-d). Suppressing autophagy would likely remove a major protective mechanism for memory T cells harboring latent HIV-1, thereby unleashing the cell death machinery triggered by viral replication and by addition of apoptosis inducers.

Despite enormous challenges, the development of HIV-1 vaccines has shown great promises in inducing immune protection against the virus. Broad-spectrum neutralization antibodies are valuable in inhibiting viremia by neutralizing HIV-1. Use of CAR T cells to target HIV-1 proteins can suppress HIV-1 infections in humanized mice or patient samples. In addition to blocking viral fusion with cell membranes and integration into host genome, broad neutralizing antibodies may be used to replace agents for ART to prevent new infections during SECH treatments.

In this example, a SECH approach was shown in a humanized mouse model to eradicate the HIV-1 reservoir by a combination of latency reversal, inhibition of autophagy, promotion of apoptosis and blocking of new rounds of viral replication by ART. Continuous SECH treatments via the oral route can safely and effectively reduce and clear HIV-1 reservoirs established in humanized mice. Moreover, treating PBMCs from HIV-1 patients by SECH led to the successful clearance of HIV-1 infections. The example shows a new strategy to treat HIV-1 infections by selectively eliminating host cells harboring replication-competent HIV-1.

Methods

Flow cytometry. The following antibodies from Biolegend were used for flow cytometry: Pacific Blue-anti-mouse CD45 (1:100, 103126, clone 30-F11,) APC-anti-human CD45 (1:100, 304012, clone HI30), Pacific Blue-anti-human CD19 (1:100, 302232, clone HIB19), PE-anti-human CD4 (1:100, 317414, clone OKT4), APC/Fire-750-anti-human CD8 (1:100, 34474, clone SK1), FITC-anti-human CD56 (1:100, 392413, clone QA17A16), Pacific Blue-anti-human CD19 (1:100, 302232, clone HIB19), Pacific Blue-anti-human CD3 (1:100, 300329, clone HIT3a), PE/Cy7-anti-HLA-HLA-DR, DP, DQ (1:100, 361708, clone Tü139), PerCP/Cy5.5-anti-human CD11b (1:100, 301327, clone ICRF44), APC/Fire-750-anti-human CD163 (1:100, 333633, clone GHI/61), PE-anti-human CD123 (1:100, 306005, clone 6H6), Alexa Fluor 488 anti-human CD11c (1:100, 301618, clone 3.9), PerCP/Cy5.5-anti-human CD3 (1:100, 300328, clone HIT3a), PerCP/Cy5.5 anti-human CD123 (1:100, 306016, clone 6H6), FITC-anti-mouse CD45 (1:100, 103108, clone 30-F11), PE/Cy7 anti-human CD197 (CCR7) (1:100, 353226, clone G043H7), PE-anti-human CD45R0 (1:100, 304244, clone UCHL1), FITC-anti-human CD45RA (1:100, 304148, clone HI100) and Alexa Fluor 488-anti-CD68 (1:30, 333812, clone Y1/82A), PerCP/Cy5.5-anti-human CD19 (1:100, 302230, clone HIB19), PE-anti-human CD27 (1:100, 356406, clone M-T271) and Pacific Blue-anti-human IgD (1:100, 348224, clone IA6-2). PE-anti-human CD3 (1:100, 556612, clone SP34) and V50-anti-human CD4 (1:100, 560345, clone RPA-T4) were from BD Biosciences. The cells stained with indicated antibodies were analyzed using a BD LSR II flow cytometer (BD Biosciences). To detect HIV-1 p24, spleen cells from Hu-HSC mice were stimulated with 5 μg/ml PHA (Sigma) and 6 ng/ml IL-2 (Biolegend). The cells were stained for T cell markers, followed by fixation and permeabilization using the Cytofix/Cytoperm buffer (BD Bioscience) and intracellular staining with PE-conjugated anti-p24 (1:30, 6604667, clone KC57, Beckman Coulter). The cells were analyzed by flow cytometry using a BD LSR II flow cytometer (BD Bioscience) and FlowJo software (version 10.5.3, BD Bioscience).

T cell isolation and infection with HIV-1. Peripheral blood mononuclear cells (PBMCs) of anonymous healthy donors from the Gulf Coast Blood Center were used to purify CD4⁺ T cells with anti-CD4 MACS beads (Miltenyi Biotec). CD3⁺CD4⁺CD45RA⁺CD45RO⁻CCR7⁺ naïve T cells, CD3⁺CD4⁺CD45RA⁻CD45RO⁺CCR7+ central memory T cells (CMT) and CD3⁺CD4⁺CD45RA⁻CD45RO⁺CCR7⁻ effector memory T cells (EMT) were sorted using a BD FACSAria flow cytometer (BD Bioscience). Naive CD4⁺ T cells were stimulated with 5 μg/ml PHA and 6 ng/ml IL-2 in RPMI complete medium for 2 days to generate activated CD4⁺ T cells. Sorted T cell subsets or activated T cells were infected with HIV-1 CXCR4-tropic NL4-3 or CCRS-tropic AD8 (both virus clones were obtained from the NIH AIDS Reagent Program) at the indicated MOI for 2 h. The cells were washed with PBS and cultured in RPMI complete medium containing 30 nM CCL19 (Biolegend) and 0.3 ng/ml IL-2 (Biolegend) and for 4 days to establish latent HIV infection as described.

For gene silencing, CMT were transfected with 100 nM siRNA targeting Atg7 or control siRNA (Dharmacon) using the Neon Transfection System at 2150 volts with one pulse of 20 mini seconds (Life Technologies). Virtually all cells were transfected using this condition with a fluorescently labeled siRNA. Gene silencing was confirmed by Western blot (FIG. 1a). The cells were infected with 0.1 MOI of HIV-1 (HIV-1 NL4-3) at 37 ° C. for 2 h. The cells were then cultured with CCL19 as above to establish HIV latent infection.

Naïve T cells, activated T cells, CMT or EMT with or without infection with HIV-1 (NL4-3, 1 MOI) were cultured for 4 days in the presence of 30 nM CCL19 and 0.3 ng/ml IL-2 for 4 days to establish latent HIV infection. The cells with or without HIV-1 infections were stimulated with 0.1 μM IDB (Ingenol-3,20-dibenzonate, ENZO Life Sciences) in the presence of 0.2 μM ABT-263 (Adooq Bioscience), 2 μM SAR405 (MedChemExpress) or 10 μM chloroquine (CQ, Sigma) as indicated for 48 h. The cells were then incubated with 1 μM FITC-DEVD-FMK (Biovision), followed by staining with APC-Annexin V (Biolegend) and intracellular staining with PE-anti-HIV p24. The cells were analyzed by flow cytometry. The percentage of cell death was calculated by the loss of live cells negative for annexin V and DEVD staining by comparing treated and untreated groups: (untreated—treated)/untreated x 100%.

To determine the specificity in the killing of HIV-1 infected cells, uninfected CD4 cells were labeled with CellTrace Violet dye (ThermoFisher Scientific) and mixed with HIV-1-infected cells at the ratio of 1:1. The cells were resuspended in RIPMI complete medium containing 0.2 μM BMS-626529, with 0.1 μM IDB, 0.2 μM ABT-263 and 2 μM SAR405 as indicated for 48 h. The cells were incubated with 1 μM FITC-DEVD-FMK, followed by staining with APC-Annexin and analyses by flow cytometry.

CD4 cells with or without HIV-1 infection cultured with or without 1 μM SAR405 for 12 hours and fixed in the 4% formaldehyde (ThermoFisher Scientific). The fixed cells were then incubated with rabbit anti-LC3A/B antibody (1:100, 12741s, Cell Signaling Technology), followed by staining with Alexa Fluor 594 donkey anti-rabbit IgG (1:5000, A21207, Thermo Fisher Scientific). LC3 punctate in the cells were quantified under a fluorescent microscope.

Detection of HIV-1 DNA products of reverse transcription and proviral DNA integration. CMT at different hours after HIV-1 infection were used for PCR to detect HIV-1 reverse transcription products as described, including early R/U5 product, sense primer (5′-GGCTAACTAGGGAACCCACTG-3′) SEQ. ID NO. 1, antisense primer (5′-CTGCTAGAGATTTTCCACACTGAC-3′) SEQ. ID NO. 2, and late LTR-gag product, sense primer (5′-CAGATATCCACTGACCTTTGG-3′) SEQ. ID NO. 3, antisense primer (5′-GCTTAATACTGACGCTCTCGCA-3′) SEQ. ID NO. 4. (3-Globin was detected by PCR with β-Globin forward, 5′-CCCTTGGACCCAGAGGTT CT-3′ (SEQ. ID NO. 5) and (3-Globin reverse, 5′-CGAGCACTTTCTTGCCATGA-3′ (SEQ. ID NO. 6). R/U5 and LTR-gag PCRs were normalized against (3-Globin. Genomic DNA from CD4⁺ T cells at 4 days after initial HIV-1 infections was used for real-time PCR for Alu-gag using the following primers: Alu forward primer (5′-GCCTCCAAAGTGCTGGGATTACAG-3′) SEQ. ID NO. 7 and gag reverse primer (5′-GTTCCTGCTATGTCACTTCC-3′) SEQ. ID NO. 8. The relative levels of Alu-gag were normalized against (3-Globin.

Western blot. PBMC CD4⁺ T cells with or without infection by HIV-1 cultured for 4 days with CCL19 as in FIG. 1a. The cells were then cultured in the absence or presence of 0.1 μM IDB for 24 h. The cells were lysed in lysis buffer containing 50 mM HEPES, pH 7.5, 150 mM NaCl, 1 mM EDTA, 1% Triton X-100, 1X protease inhibitor mixture (Roche Applied Science) and 10 μM Benzyloxycarbonyl-Val-Ala-Asp (OMe) fluoromethylketone (zVAD-FMK, ENZO Life Sciences). The cell lysates were determined by the Bradford assay (Bio-Rad). Samples were used for SDS-PAGE and Western blot analyses by probing with different antibodies: monoclonal antibodies to caspase-9 (1:1,000, M054-3, clone 5B4) and caspase-7 (1:1,000, M053-3, clone 4G2) from Medical & Biological Laboratories; polyclonal rabbit antibodies to Atg-7 (1:1,000, 2631S), cleaved caspase-9 (1:1,000, 52873s), caspase-3 (1:1,000, 9665s); cleaved caspase-3 (1:1,000, 9501s), caspase-6 (1:1,000, 9762s), Mc1-1 (1:1,000, 5453s), Bcl-2 (1:1,000, 4223s), Bcl-xL (1:1,000, 2762s), Bak (1:1,000, 12105s), Bax (1:1,000, 2772s), LC3 (1:1000, 4108s) from Cell Signaling Technology and monoclonal antibody to β-Actin (1:50,000, sc-47778, clone C4) from Santa Cruz Biotechnology. The blots were them incubated with HRP-conjugated goat anti-Mouse IgG1 (1:50,000, 1070-05, Southern Biotech) or HRP-conjugated goat anti-Rabbit IgG (1:50,000, ab6721, Abcam) and developed using SuperSignal West Dura Extended Duration Substrate (ThermoFisher).

Quantification of HIV-1 mRNA and DNA. HIV-1 mRNA was measured by RT-PCR similar to the described protocols. LTR-GAG was amplified with forward primer (LTR-GAG-AF), 5′-GATCTCTCGACGCAGGACTC-3′ (SEQ. ID NO. 9) and reverse primer (LTR-GAG-AR), 5′-CGCTTAAT ACCGACGCTCTC-3′ (SEQ. ID NO. 10), and detected with the LTR-GAG probe, SHEX/CCAGTCGCC/ZEN /GCCCCTCGCCTC/3IABkFQ (SEQ. ID NO. 11). HIV-1 pol-1 was amplified with POL-1 forward primer (POL-1-AF), 5′-AGCAGGAAGATGGCCAGTAA-3′ (SEQ. ID NO. 12) and reverse primer (POL-1-AR), 5′-GGATTGTAGGGAATGCCAAA-3′ (SEQ. ID NO. 13), and detected with the pol-1 probe FAM/CCCACCAAC/ZEN/ARGCRGCCTTAACYG/3IABKFQ (SEQ. ID NO. 14) in iTaq Universal Probes Supermix (Bio-Rad). The reaction was carried out by iTaq Universal Probes Supermix (Bio-Rad) with a QuantStudio 5 Real-Time PCR System and analyzed with the QuantStudio Design and Analysis Software (Applied Biosystem). Cycle threshold (Ct) values were calibrated using standard curve generated with the standard samples with known amounts of viral copies.

Spleen cells were isolated and red blood cells were lysed with ammonium chloride lysing buffer (0.15 M NH4Cl, 10 mM KHCO3, 0.1 mM EDTA). Bone marrow cells were collected from femur and tibia. The cells were used for mRNA preparation with MagMAX-96 for Microarrays Total RNA Isolation Kit (ThermoFisher Scientific). Tissues (50 mg) were homogenized with Precellys Lysing Kit (Cayman Chemical). mRNA was extracted and converted to cDNA using the SuperScript VILO cDNA Synthesis Kit (Thermo Fisher Scientific), followed by real-time PCR for Pol-1 and LTR-gag as above. Quantity of viral DNA was performed by real time PCR of genomic DNA from the cells with the described primers as described⁷⁰: forward primer, 5′-GGTCTCTCTGGTTAGACCAGAT-3′ (SEQ. ID NO. 15) and reverse primer, AGATTTTCCACACTG (SEQ. ID NO. 16) and probe 5′-6FAMAGTAGTGTGTGCCCGTCTGTT-TAMRA-3′) SEQ. ID NO. 17 for amplification of an HIV-1 LTR sequence.

Generation of humanized mice for HIV-1 infection and cure studies. NSG-SGM3 mice (Stock No: 013062, The Jackson Laboratory) were maintained on a 12-hour light/dark cycle with the temperature (22° C.) and humanity (40-60%) controlled environment in the specific-pathogen-free barrier animal facility at the Houston Methodist Research Institute. Newborn male and female mice were injected intrahepatically with CD34⁺ human stem cells (5×10⁴/mouse; AllCells LLC). Three months later, reconstitution of human immune cells in mouse peripheral blood was determined by flow cytometry. Both male and female mice were used for the experiments. HIV-1-infection and cure experiments were performed in Biosafety Level 2 facilities in the Houston Methodist Research Institute. These human CD34⁺ cell-reconstituted mice (Hu-HSC mice) were then injected intraperitoneally with HIV-1 AD8 (1000 pfu/mouse). Seven days later, peripheral blood was collected and RNA was extracted using the MagMAX-96 Blood RNA Isolation Kit (Thermo Fisher Scientific). RNA was converted to cDNA using the SuperScript VILO cDNA Synthesis Kit (Thermo Fisher Scientific), followed by real-time PCR for Pol-1 and LTR-gag as above. Experiments were performed according to federal and institutional guidelines and with the approval of the Institutional Animal Care and Use Committee of the Houston Methodist Research Institute.

A total of 8 sets of experiments were performed to establish HIV-1 infections and treatments using HSC-Hu mice, including 3 sets of experiments to test the reconstitution of NSG-SGM3 mice with human CD34⁺ stem cells and the establishment of HIV-1 infections in these mice, 2 sets of experiments to determine the dose of drugs that could be safely used in C57BL/6 mice and NSG-SGM3-derived HSC-Hu mice, one set of experiments in FIG. 12c-g to test the SECH procedure after initial ART treatments, one set of experiments with SECH treatments in FIGS. 3-4 and FIGS. 11-14, and one set of SECH treatments with or without JQ1 in FIG. 5 and FIG. 15. For SECH treatments, raltegravir (20 mg/kg b.w.), BMS-663068 (20 mg/kg b.w., Adooq Bioscience), IDB (2.5 mg/kg b.w.), ABT-263 (50 mg/kg b.w.) and SAR405 (50 mg/kg b.w.) with or without JQ1 (25 mg/kg b.w.) were formulated in the solvent containing 10% ethanol, 30% polyethylene glycol 400 (Sigma) and 60% Phosal 50 PG (Fisher Scientific), and administered by oral gavage once every two days. Raltegravir and BMS-663068 (20 mg/kg b.w.) alone were also administered on the alternate days. For the ART control group, raltegravir and BMS-663068 (20 mg/kg b.w.) were given daily. In addition, tablets containing non-steroid anti-inflammatory carprofen (2 mg in each 5 g tablet, Bio-Serv) were supplied together with regular diet pellets to mice. During treatments, mice were monitored daily for body weight, food consumption and mobilities. At the end of experiment, histological analysis by H&E staining was carried out in the major vital organs (brain, liver, lung and kidney).

Peripheral blood was collected at different intervals to detect HIV-1 mRNA by RT-PCR. After treatments, spleen and bone marrow were collected for analyses by RT-PCR, virus outgrowth assay and intracellular staining for HIV-1 p24. Some mice were kept for an additional two months with no treatments. Virus clearance was determined in the spleen, bone marrow by RT-PCR, TZA assay and p24 intracellular staining. RT-PCR was also performed to detect HIV-1 mRNA in the lung, liver and kidney for some mice.

TZA assay. TZM-bl cells obtained from the NIH AIDS Reagent Program were cultured in 96 well plates (60,000 cells/well) for 24 hours. TZA assay was performed similar to described procedures. Spleen and bone marrow cells (5×10⁶/sample) from Hu-HSC mice were stimulated with anti-CD3- and anti-D28-Dynalbeads for 48 h, followed by co-cultured with TZM-bl cells for another 48 h in the presence of 5 μg/ml PHA, 0.1 μg/ml LPS and 100 nM CpG. Beta-galactosidase activity was determined using the Beta-Glo Assay System (Promega). This virus outgrowth assay can detect between 1 and 400 pfu of HIV-1 in a linear fashion, and the virus titers in the samples were calculated based on HIV-1 standard titration.

Hu-HSC mouse-based virus outgrowth assay in vivo (hmVOA). Spleen or bone marrow cells (5×10⁶/sample) from HIV-1-infected Hu-HSC mice treated by SECH or ART were transferred into uninfected recipient Hu-HSC mice intravenously similar to previously described procedures. HIV-1 in the peripheral blood of recipient mice was determined 4 weeks later by RT-PCR. PBMCs from HIV-1-infected patients (3×10⁶/sample) with or without SECH treatments in vitro were also were transferred into Hu-HSC mice intravenously. HIV-1 in the mouse peripheral blood was determined by RT-PCR.

Treatment of PBMCs from HIV-1-infected patients. PBMCs from HIV-1 patients were resuspended in RPMI complete medium containing and 0.3 ng/ml IL-2 and 50 ng/ml M-CSF and cultured with 0.2 μM BMS-626529, 0.2 μM raltegravir, 25 nM IDB, 20 nM ABT-263, 0.1 μM SAR405 and 0.25 μM JQ1 for 2 days as one cycle of treatments. The cells were then washed and cultures in the same medium for next cycle of culture. After 5 cycles of culture, RNA was prepared from the cells for RT-PCR analyses of HIV-1. For hmVOA, the cells were incubated with biotin-conjugated anti-CD8 and BioMag streptavidin beads (Sigma) to deplete CD8⁺ T cells. The cells (3×10⁶) were then adoptively transfer into uninfected Hu-HSC mice intravenously for hmVOA as above. Experiments with ART-naïve patients were performed according to federal and institutional guidelines, with written informed consents and the approval of the Institutional Review Boards of University of Texas Health Science Center at Houston and Houston Methodist Research Institute. Experiments with de-identified samples from ART-treated patients were performed according to federal and institutional guidelines with the approval of the Institutional Review Board of the Houston Methodist Research Institute.

Statistical analyses. GraphPad Prism 8 was used for statistical analyses. Data were presented as the mean ±SD and P values were determined by two-tailed Student's t-test. Mann—Whitney test was also used for analyzing humanized mice and patient PBMCs. Significant statistical differences (P<0.05 or P<0.01) are indicated.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating or inhibiting human immunodeficiency virus (HIV) in a subject, comprising:
a) reactivating latent HIV integrated into the genome of a cell infected with HIV in the subj ect,
b) administering to the subject an effective amount of a therapeutic agent to inhibit HIV infection, and
c) administering to the subject an effective amount of a therapeutic agent to eliminate or reduce the number of cells containing replication-competent HIV, wherein the therapeutic agent inhibits autophagy and promotes apoptosis of infected cells in the subject.

2. (canceled)

3. The method of claim 1, wherein step a) reactivating latent HIV comprises contacting the cell infected with HIV with a protein kinase activator, preferably a protein kinase C activator; a glycogen synthase kinase 3 inhibitor; a bromodomain inhibitor; a histone deacetylase (HDAC) inhibitor; a histone acetyltransferase (HAT) inhibitor; a noncanonical NF-kB activator; an epigenetic modifier (e.g., JQ1D or CPI-203); a toll-like receptor (TLR) agonist; a cytokine; inhibitor of apoptosis (IAP) antagonist (IAP inhibitor e.g., AZD5582); or a combination thereof.

4. The method of claim 1, wherein step a) comprises contacting the cell infected with HIV with a protein kinase C activator selected from the group consisting of ingenol-3,20-dibenzoate; prostratin;
bryostatin; a salt, ester, or prodrug thereof; and combinations thereof.

5. The method of claim 1, wherein step a) comprises contacting the cell infected with HIV with ingenol-3,20-dibenzoate in an amount from 0.2 mg/kg to 10 mg/kg body weight.

6. The method of claim 1, wherein step a) reactivating latent HIV comprises contacting the cell infected with HIV with a protein kinase activator and a bromodomain inhibitor.

7. The claim 6, wherein the protein kinase C activator comprises ingenol-3,20-dibenzoate and the bromodomain inhibitor comprises includes JQ1.

8-9. (canceled)

10. The method of claim 1, wherein the therapeutic agent to inhibit HIV infection in step b) comprises a therapeutic agent that inhibits viral replication, viral protease activity, viral reverse transcriptase activity, viral entry into a cell, viral integrase activity, viral Rev activity, viral Tat activity, viral Nef activity, viral Vpr activity, viral Vpu activity, or viral Vif activity, an anti-HIV broad neutralization antibody, an anti-HIV CAR T cell, or a combination thereof.

11. The method of claim 1, wherein the therapeutic agent to inhibit HIV infection in step b) comprises a therapeutic agent that inhibits viral entry into cells (e.g., CD4⁺ T cells); inhibits viral integration into cells; or a combination thereof.

12. The method of claim 1, wherein the therapeutic agent to inhibit HIV infection in step b) is selected from the group consisting of BMS-626529; raltegravir; a salt, ester, or prodrug thereof; and combinations thereof.

13. The method of claim 1, wherein the therapeutic agent to inhibit HIV infection in step b) is selected from the group consisting of BMS-626529 and its prodrug BMS-663068.

14. The method of claim 1 any one of the preceding claims, wherein step b) comprises administering BMS-626529 or BMS-663068 in an amount from 2 mg/kg to 100 mg/kg body weight or raltegravir in an amount from 2 mg/kg to 100 mg/kg body weight.

15. (canceled)

16. The method of claim 1, wherein the therapeutic agent to eliminate or reduce the number of cells containing replication-competent HIV in step c) is selected from a therapeutic agent that inhibits anti-apoptotic molecules (e.g., inhibits BCL-2); inhibits autophagy; or a combination thereof.

17. The method of claim 1, wherein cells containing replication-competent HIV in step c) are selected from CD4⁺ T cells, macrophages, dendritic cells, or combinations thereof.

18. The method of claim 1, wherein the therapeutic agent to eliminate or reduce the number of cells containing replication-competent HIV in step c) inhibits autophagy.

19. (canceled)

20. The method of claim 1, wherein the therapeutic agent to eliminate or reduce the number of cells containing replication-competent HIV in step c) inhibits anti-apoptotic molecules.

21. The method of claim 1, wherein the therapeutic agent to eliminate or reduce the number of cells containing replication-competent HIV in step c) comprises a BCL-2 inhibitor such as navitoclax (ABT-263), ABT-119, or venetoclax (RG7601 or GDC-0199); a MCL-1 inhibitor such as AZD5991, S63845, A-121-477, or AMG 176; SAR-405; chloroquine; Spautin-1; SBI-0206965; MRT68921; an agent that silences expression of an autophagy gene, such as Atg7; or a combination thereof.

22. The method of claim 1, wherein step c) comprises administering navitoclax in an amount from 2 mg/kg to 100 mg/kg body weight or SAR-405 in an amount from 2 mg/kg to 100 mg/kg body weight.

23. (canceled)

24. The method of claim 1, wherein the method comprises administering IDB, ABT-263, and SAR405 or chloroquine.

25-29. (canceled)

30. A kit for treating or inhibiting human immunodeficiency virus (HIV) in a subject, comprising:
an effective amount of a therapeutic agent for reactivating latent HIV integrated into the genome of a cell infected with HIV in the subject,
optionally an effective amount of a therapeutic agent to inhibit HIV infection, and an effective amount of a therapeutic agent to eliminate or reduce the number of cells containing replication-competent HIV.

31-59. (canceled)


SEQUENCE LISTING (17 sequences)

SEQ ID NO: 1 - DNA, 21 bp, Artificial Sequence
  Synthetic construct
ggctaactagggaacccactg

SEQ ID NO: 2 - DNA, 24 bp, Artificial Sequence
  Synthetic construct
ctgctagagattttccacactgac

SEQ ID NO: 3 - DNA, 21 bp, Artificial Sequence
  Synthetic construct
cagatatccactgacctttgg

SEQ ID NO: 4 - DNA, 22 bp, Artificial Sequence
  Synthetic construct
gcttaatactgacgctctcgca

SEQ ID NO: 5 - DNA, 20 bp, Artificial Sequence
  Synthetic construct
cccttggacccagaggttct

SEQ ID NO: 6 - DNA, 20 bp, Artificial Sequence
  Synthetic construct
cgagcactttcttgccatga

SEQ ID NO: 7 - DNA, 24 bp, Artificial Sequence
  Synthetic construct
gcctccaaagtgctgggattacag

SEQ ID NO: 8 - DNA, 20 bp, Artificial Sequence
  Synthetic construct
gttcctgctatgtcacttcc

SEQ ID NO: 9 - DNA, 20 bp, Artificial Sequence
  Synthetic construct
gatctctcgacgcaggactc

SEQ ID NO: 10 - DNA, 20 bp, Artificial Sequence
  Synthetic construct
cgcttaataccgacgctctc

SEQ ID NO: 11 - DNA, 21 bp, Artificial Sequence
  Synthetic construct
ccagtcgccgcccctcgcctc

SEQ ID NO: 12 - DNA, 20 bp, Artificial Sequence
  Synthetic construct
agcaggaagatggccagtaa

SEQ ID NO: 13 - DNA, 20 bp, Artificial Sequence
  Synthetic construct
ggattgtagggaatgccaaa

SEQ ID NO: 14 - DNA, 24 bp, Artificial Sequence
  Synthetic construct
cccaccaacargcrgccttaacyg

SEQ ID NO: 15 - DNA, 22 bp, Artificial Sequence
  Synthetic construct
ggtctctctggttagaccagat

SEQ ID NO: 16 - DNA, 15 bp, Artificial Sequence
  Synthetic construct
agattttccacactg

SEQ ID NO: 17 - DNA, 21 bp, Artificial Sequence
  Synthetic construct
agtagtgtgtgcccgtctgtt